SEQUENCE LISTING (8 sequences)

SEQ ID NO: 1 - DNA (genomic), 29 bp; unknown-stranded, unknown
atgaattcaagttctactcgcgtaggcgc

SEQ ID NO: 2 - DNA (genomic), 29 bp; unknown-stranded, unknown
atgaattcccgaagatgtctccaggatgt

SEQ ID NO: 3 - DNA (genomic), 27 bp; unknown-stranded, unknown
atgaattcaagggtcagtgtggagagt

SEQ ID NO: 4 - DNA (genomic), 32 bp; unknown-stranded, unknown
ttgaattcgtgtgggtactctccacactgacc

SEQ ID NO: 5 - DNA (genomic), 28 bp; unknown-stranded, unknown
atgaattcctgcctcctccgatggcatt

SEQ ID NO: 6 - DNA (genomic), 32 bp; unknown-stranded, unknown
atgaattcgcctccaggaatgaagggagaagt

SEQ ID NO: 7 - DNA (genomic), 34 bp; unknown-stranded, unknown
atgaattcgttccagcagccttggactggtaagc

SEQ ID NO: 8 - DNA (genomic), 29 bp; unknown-stranded, unknown
atgaattcgccagtcccaggttagaggca

(19) United States
(12) Patent Application Publication
(10) Pub. No.: US 2002/0142391 A1
(43) Pub. Date: Oct. 3, 2002

(54) Synthesis of human procollagens and collagens in recombinant DNA systems

(76) Inventors: Kari I. Kivirikko, Oulu (FI); Taina Pihlajaniemi, Oulunsalo (FI)

Correspondence Address: Intellectual Property Department, FibroGen, Inc., 225 Gateway Blvd., South San Francisco, CA, 94080, US

(21) Appl. No.: 10/040,204

(22) Filed: Dec. 19, 2001

Related U.S. Application Data
(63) Continuation of application No. 09/260,582, filed on Mar. 1, 1999, now abandoned.
Application No. 09/260,582 is a continuation-in-part of application No. 08/211,820, filed on Aug. 11, 1994.
Application No. 09/260,582 is a continuation-in-part of application No. 08/486,860, filed on Jun. 7, 1995, now abandoned.
Application No. PCT/US92/09061 is a continuation-in-part of application No. 07/780,899, filed on Oct. 23, 1991, now abandoned.
Application No. 08/486,860 is a continuation-in-part of application No. 08/210,063, filed on Mar. 16, 1994.
Application No. 08/210,063 is a 371 of international application No. PCT/US92/22333, filed on Jun. 10, 1992.
Application No. PCT/US92/22333 is a continuation of application No. 07/713,945, filed on Jun. 12, 1991, now abandoned.
(60) Provisional application No. 60/006,608, filed on Nov. 13, 1995.

(51) Int. Cl. C12P 21/02; C12N 1/18; C12N 5/06; C12N 5/04

(52) U.S. Cl. 435/69.1; 435/325; 435/254.2; 435/410; 435/348

(57) ABSTRACT

Methods of making collagen with hosts, and vectors that express collagen, and collagen post-translation enzymes are disclosed. Collagen post-translation enzymes include prolyl-4-hydroxylase, lysyl hydroxylase, lysyl oxidase, C-proteinase, and N-proteinase, and these enzymes increase the yield of properly folded, recombinant collagen in non-mammalian hosts. The collagens produced by these methods, hosts, and vectors include both homotrimer and heterotrimer collagen made from single or multiple collagen genes, respectively.

This application is a continuation-in-part of U.S. applications, Ser. No. 08/211,820, filed Aug. 11, 1994 (“the '820 application”), Ser. No. 08/486,860, filed Jun. 7, 1995 (“the '860 application”), and provisional U.S. application, Ser. No. 60/006,608, filed Nov. 13, 1995. The '820 application is a U.S. National application, pursuant to 35 U.S.C. § 371, of PCT Application Ser. No. PCT/US92/09061, filed Oct. 22, 1992, which is a continuation-in-part of U.S. application No. 07/780,899, filed Oct. 23, 1991, now abandoned. The '860 application is a continuation-in-part of the '820 application and U.S. application Ser. No. 08/210,063, filed Mar. 16, 1994, which is a U.S. National application, pursuant to 35 U.S.C. § 371, of PCT Application Ser. No. PCT/US92/22333, filed Jun. 10, 1992, which is a continuation of U.S. application Ser. No. 07/713,945, filed Jun. 12, 1991, now abandoned. Each of these applications is incorporated herein by reference.

Portions of the invention described herein were made in the course of research supported in part by NIH grants AR38188 and AR39740. The Government may have certain rights in this invention.

### FIELD OF THE INVENTION

The present invention is directed to the recombinant production of procollagen, collagen and fragments thereof.

### BACKGROUND OF THE INVENTION

The ExtraCellular Matrix. The most abundant component of the extracellular matrix is collagen. Collagen molecules are generally the result of the trimeric assembly of three polypeptide chains containing, in their primary sequence, (-Gly-X-Y-)n repeats which allow for the formation of triple helical domains (van der Rest et al. FASEB J. 5:2814-2823 (1991)).

During their biosynthesis, collagens undergo various post-translational modifications (Van der Rest et al., Adv. Mol. Cell Biol. 6:1-67 (1993)). For example, the proline residues of collagen are hydroxylated into 4-hydroxyproline, thereby contributing to the stability of collagen by allowing the formation of additional interchain hydrogen bonds. The enzyme catalyzing this modification is prolyl 4-hydroxylase (Kivirikko et al., Post-translational modifications of Proteins (Harding, J. J., Crabbe, M. J. C., eds) pp. 1-51, CRC Press, Boca Raton, Fla. (1992)). As further example, the N-propeptide and C-propeptide comprising the collagen precursor molecule, “procollagen,” are cleaved during post-translational events by the enzymes N-proteinase and C-proteinase, respectively.

As a consequence of the diverse structural and functional properties of collagen in its various forms or “types,” collagen can contribute significantly to the high diversity of the extracellular matrix.

Collagen Types. Nineteen distinct collagen types have been identified in vertebrates. These collagen types are numbered by Roman numerals and the chains found in each collagen type are identified with Arabic numerals. A detailed description of structure and biological functions of the various different types of naturally occurring collagens can be found, among other places, in Ayad et al., The Extracellular Matrix Facts Book, Academic Press, San Diego, Calif.; Burgeson, R. E., and Nimmi, “Collagen types: Molecular Structure and Tissue Distribution,” Clin. Orthop. 282:250-272 (1992); Kielty, C. M. et al., “The Collagen Family: Structure, Assembly And Organization In The Extracellular Matrix,” in Connective Tissue And Its Heritable Disorders, Molecular Genetics, And Medical Aspects, Royce, P. M. and Steinmann, B., Eds., Wiley-Liss, NY, pp. 103-147 (1993).

Type I collagen is the major fibrillar collagen of bone and skin. Type I collagen is a heterotrimeric molecule comprising two α1(I) chains and one α2(I) chain. Details on preparing purified type I collagen can be found, among other places, in Miller et al., Methods In Enzymology 82:33-64 (1982), Academic Press.

Type II collagen is a homotrimeric collagen comprising three identical α1(II) chains Purified Type II collagen may be prepared from tissues by, among other methods, the procedure described in Miller et al., Methods In Enzymology, 82:33-64 (1982), Academic Press.

Type III collagen is a major fibrillar collagen found in skin and vascular tissues. Type III collagen is a homotrimeric collagen comprising three identical α1(III) chains. Methods for purifying type III collagen from tissues can be found in, among other places, Byers et al., Biochemistry 13:5243-5248 (1974) and Miller et al., Methods in Enzymology 82:33-64 (1982), Academic Press.

Type IV collagen is found in basement membranes in the form of a sheet rather than fibrils. The most common form of type IV collagen contains two α1(IV) chains and one α2(IV) chain. The particular chains comprising type IV collagen are tissue-specific. Type IV collagen may be purified by, among other methods, the procedures described in Furuto et al., Methods in Enzymology 144:41-61 (1987), Academic Press.

Type V collagen is a fibrillar collagen found in, primarily, bones, tendon, cornea, skin, and blood vessels. Type V collagen exists in both homotrimeric and heterotrimeric forms. One type of type V collagen is a heterotrimer of two α1(V) chains and α2(V). Another type of type V collagen is a heterotrimer of α1(V), α2(V), and α3(V). Yet another type of type V collagen is a homotrimer of α1(V). Methods for isolating type V collagen from natural sources can be found, among other places, in Elstrow et al., Collagen Rel. Res. 3:181-193 (1983) and Abedin et al., Biosci. Rep. 2:493-502 (1982).

Type VI collagen has a small triple helical region and two large non-collagenous remainder portions. Type VI collagen is a heterotrimer comprising α1(VI), α2(VI), and α3(VI) chains. Type VI collagen is found in many connective tissues. Descriptions of how to purify type VI collagen from natural sources can be found, among other places, in Wu et al., Biochem. J. 248:373-381 (1987), and Kielty, et al., J. Cell Sci. 99:797-807.

Type VII collagen is a fibrillar collagen found in particular epithelial tissues. Type VII is a homotrimeric molecule of three α1(VII) chains. Descriptions of how to purify type VII collagen from tissue can be found in, among other places, Lundstrom et al., J. Biol. Chem. 261:9042-9048 (1986), and Bentz et al., Proc. Natl. Acad. Sci. USA 80:3168-3172 (1983).

Type VIII collagen can be found in Descemet's membrane in the cornea. Type VIII collagen is a heterotrimer comprising two α1(VIII) chains and one α2(VIII) chain, although other chain compositions have been reported. Methods for the purification of type VIII collagen from nature can be found, among other places, in Benya et al., J. Biol. Chem. 261:4160-4169 (1986), and Kapoor et al., Biochemistry 25:3930-3937 (1986).

Type IX collagen is a fibril associated collagen which can be found in cartilage and vitreous humor. Type IX collagen is a heterotrimeric molecule comprising α1(IX), α2(IX), and α3(IX) chains. Procedures for purifying type IX collagen can be found, among other places, in Duance, et al., Biochem. J. 221:885-889 (1984), Ayad et al., Biochem. J. 262:753-761 (1989), Grant et al., The Control of Tissue Damage, Glauert, A. M., Ed., El Sevier, Amsterdam, pp. 3-28 (1988).

Type X collagen is a homotrimeric compound of α1(X) chains. Type X collagen has been isolated from, among other tissues, hypertrophic cartilage found in growth plates.

Type XI collagen can be found in cartilaginous tissues associated with type II and type IX collagens, as well as other locations in the body. Type XI collagen is a heterotrimeric molecule comprising α1(XI), α2(XI), and α3(XI) chains. Methods for purifying type XI collagen can be found, among other places, in Grant et al., In The Control of Tissue Damage, Glauert, A. M., ed., El Savier, Amsterdam, pp. 3-28 (1988).

Type XII collagen is a fibril associated collagen found primarily associated with type I collagen. Type XII collagen is a homotrimeric molecule comprising three α1(XII) chains. Methods for purifying type XII collagen and variants thereof can be found, among other places, in Dublet et al., J. Biol. Chem. 264:13150-13156 (1989), Lundstrum et al., J. Biol. Chem. 267:20087-20092 (1992), Watt et al., J. Biol. Chem. 267:20093-20099 (1992).

Type XIII is a non-fibrillar collagen found, among other places, in skin, intestine, bone, cartilage, and striated muscle. A detailed description of the type XIII collagen may be found, among other places, in Juvonen et al. J. Biol. Chem. 267:24700-24707 (1992).

Type XIV is a fibril associated collagen. Type XIV collagen is a homotrimeric molecule comprising three α1(XIV) chains. Methods for isolating type XIV collagen can be found, among other places, in Aubert-Foucher et al., J. Biol. Chem. 266:19759-19764 (1992) and Watt et al., J. Biol. Chem. 267:20093-20099 (1992).

Type XV collagen is homologous in structure to type XVIII collagen. Information about the structure and isolation of natural type XV collagen can be found, among other places, in Myers et al., Proc. Natl. Acad. Sci. USA 89:10144-10148 (1992), Huebner et al., Genomics 14:220-224 (1992), Kivirikko et al., J. Biol. Chem. 269:4773-4779 (1994), and Muragaki, J. Biol. Chem. 264:4042-4046 (1994). Type XVI collagen is a fibril associated collagen, found in skin, lung fibroblast, keratinocytes, and elsewhere. Information on the structure of type XVI collagen and the gene encoding type XVI can be found, among elsewhere, in Pan et al., Proc. Natl. Acad. Sci. USA 1989:6565-6569 (1992), and Yamaguchi et al., J. Biochem. 112:856-863 (1992).

Type XVII collagen is a hemidesmosal transmembrane collagen. Information on the structure of type XVII collagen and the gene encoding type XVII collagen can be found, among elsewhere, in Li et al., J. Biol. Chem. 268(12):8825-8834 (1993), and McGrath et al., Nat. Genet. 11(l):83-86 (1995).

Type XVIII collagen is similar in structure to type XV collagen and can be isolated from the liver. Descriptions of the structures and isolation of type XVIII collagen from natural sources can be found, among other places, in Rehn et al., Proc. Natl. Acad. Sci USA 91:4234-4238 (1994), Oh et al., Proc. Natl. Acad. Sci USA 91:4229-4233 (1994), Rehn et al., J. Biol. Chem. 269:13924-13935 (1994), and Oh et al., Genomics 19:994-999 (1994).

Type XIX collagen's gene structure classify it as another member of the FACIT collagenous family. Type XIX mRNA was recently isolated from rhabdomyosarcoma cell. Descriptions of the structures and isolation of type XIX collagen can be found, among other places, in Inoguchi et al., J. Biochem. 117:137-146 (1995), Yoshioka et al., Genomics 13:884-886 (1992), Myers et al., J. Biol. Chem. 289:18549-18557 (1994).

Post-Translational Enzymes. Prolyl 4-hydroxylase is an important post-translational enzyme necessary for the synthesis of procollagen or collagen by cells. The enzyme is required to hydroxylate prolyl residues in the Y-position of the repeating -Gly-X-Y- sequences to 4-hydroxyproline. Prockop et al., N. Enql. J. Med. 311:376-386 (1984). Unless an appropriate number of Y-position prolyl residues are hydroxylated to 4-hydroxyproline by prolyl 4-hydroxylase, the newly synthesized chains cannot fold into a triple-helical conformation at 37° C. Moreover, if the hydroxylation does not occur, the polypeptides remain non-helical, are poorly secreted by cells, and cannot self-assemble into collagen fibrils.

Prolyl-4-hydroxylase from vertebrates is an α₂β₂ tetramer. Berg et al., J. Biol. Chem. 248:1175-1192 (1973); Tuderman et al., Eur. J. Biochem. 52:9-16 (1975). The α subunits (_{˜}63 kDa) contain the catalytic sites involved in the hydroxylation of prolyl residues but are insoluble in the absence of β subunits. The β subunits (_{˜}55 kDa) were found to be identical to the protein disulfide isomerase, which catalyzes thiol/disulfide interchange in a protein substrate, leading to the formation of the set of disulfide bonds which permit establishment of the most stable state of the protein. The β subunits retain 50% of protein disulfide isomerase activity when part of the prolyl-4-hydroxylase tetramer. Pihlajaniemi et al., Embo J. 6:643-649 (1987); Parkkonen et al., Biochem. J. 256:1005-1011 (1988); Koivu et al., J. Biol. Chem. 262:6447-6449 (1987)). Recently, active recombinant human enzyme has been produced in insect cells by simultaneously expressing the α and β subunits in Sf9 cells. Vuori, et al., Proc. Natl. Acad. Sci. USA 89:7467-7470 (1992).

In addition to prolyl-4-hydroxylase, other collagen post-translational enzymes have been identified and reported in the literature, including C-proteinase, N-proteinase, lysyl oxidase, and lysyl hydroxylase.

Attempts to Express Collagen. Expression of many exogenous genes is readily obtained in a variety of recombinant host-vector systems. Expression, however, becomes difficult to obtain if the final formation of the protein requires extensive post-translational processing. This is the likely reason that, prior to the present invention, expression of properly formed collagen in a fully recombinant system has not been reported. See Prockop et al., N. Enql. J. Med. 311:376-386 (1984).

Notably, rescue experiments in two different systems that synthesized only one of the two chains for type I procollagen have been reported. Specifically, it was found that a gene for the human fibrillar procollagen proα1(I) chain, the COL1A1 gene, can be expressed in mouse fibroblasts and the chains used to assemble molecules of type I procollagen, the precursor of type I collagen. However, the reports are limited to the proαa2(I) chains of mouse origin. Hence, the type I procollagen synthesized is a hybrid molecule of human and mouse origin.

Similarly, expression of a rat exogenous proα2(I) gene to generate type I rat procollagen have been reported. Thus, synthesis of a recombinant procollagen molecule in which all three chains are derived from exogenous genes was not obtained in the art.

Failure to obtain expression of genes for human collagens has made it impossible to prepare human procollagens and collagens that have a number of therapeutic uses in man and that will not produce the undesirable immune responses that have been encountered with use of collagen from animal sources. Also, many types of collagen are only available in trace quantities in tissues and can only be obtained in significant quantities by recombinant production.

### SUMMARY OF THE INVENTION

Methods. The present invention comprises the expression of at least one nucleic acid sequence encoding a collagen chain, and at least one nucleic acid sequence encoding a collagen post-translational enzyme.

More specifically, the present invention provides for methods of expressing at least a single procollagen or collagen gene (or other nucleic acid molecule) or a number of different procollagen or collagen genes (or other nucleic acid molecule) within a cell. Further, it is contemplated that there can be one or more copies of a single procollagen or collagen gene (or other nucleic acid molecule) or of the number of different such genes introduced into cells (i.e., transformation or transduction) and expressed. The present invention provides that these cells can be transformed or transfected with nucleic acids encoding collagen and enzymes that modify collagen so that they express at least one human procollagen or collagen chain that will assemble into a homotrimer or heterotrimer procollagen or collagen.

In one embodiment of the present invention, the method utilizes a procollagen or collagen gene (or other nucleic acid molecule) transfected into and expressed within cells which are a mutant, variant, hybrid or recombinant gene (or other nucleic acid molecule). Such mutant, variant, hybrid or recombinant gene may include, for example, a mutation which provides unique restriction sites for cleavage of the hybrid gene.

In a further embodiment of the present invention, such mutations provide one or more unique restriction sites do not alter the amino acid sequence encoded by the nucleic acid molecule, but merely provide unique restriction sites useful for manipulation of the molecule. Thus, the modified molecule would be made up of a number of discrete regions, or D-regions, flanked by unique restriction sites. These discrete regions of the molecule are herein referred to as cassettes. For example, cassettes designated as D1 through D4.4 are shown in FIG. 4. Molecules formed of multiple copies of a cassette are another variant of the present gene which is encompassed by the present invention. Recombinant or mutant nucleic acid molecules or cassettes which provide desired characteristics such as resistance to endogenous enzymes such as collagenase are also encompassed by the present invention.

A novel feature of the methods of the invention is that relatively large amounts of a human procollagen or collagen can be synthesized in a recombinant cell culture system that does not make any other procollagen or collagen. Systems that make other procollagens or collagens are preferred because of the extreme difficulty of separating the product of the endogenous genes for procollagen or collagen from recombinant collagen products. Using methods of the present invention, purification of human procollagen is greatly facilitated. Moreover, it has been demonstrated that the amounts of protein synthesized by the methods of the present invention are high relative to other systems used in the art.

Other novel features of the methods of the present invention are that procollagens synthesized are correctly folded proteins so that they exhibit the normal triple-helical conformation characteristic of procollagens and collagens. Therefore, the procollagens can be used to generate stable collagen by cleavage of the procollagens with proteases.

The present invention provides methods for the production of procollagens or collagens derived solely from transformed or transfected procollagen and collagen genes, such methods are not limited, however, to the production of procollagen and collagen derived solely from transformed or transfected genes.

Vectors. The present invention is also directed to vectors and plasmids used in the methods of the invention. Such vectors and/or plasmids are comprised of the nucleic acid sequence encoding the desired procollagens and collagens and necessary promoters, and other sequences necessary for the proper expression of such procollagens and collagens. In a preferred embodiment, the vectors and plasmids of the present invention further include at least one sequence encoding one or more post-translational enzymes.

In a preferred embodiment, baculoviruses are used to introduce the nucleic acids of the present invention into insect cells to effect the large-scale production of various recombinant collagens. The proteins produced in this expression system are usually correctly processed, properly folded and disulfide bonded (Luckow, V. A. and Summers, M. D., (1989), Viroloqy 170:31-39; Gruenwald, S. and Heitz, J., (1993), “Baculovirus Expression Vector System; Procedures & Methods Manual,” Pharmingen).

It is an object of the invention to construct expression vectors for various host cells that contain collagen genes from human and other sources, and to construct expression vectors that contain various collagen post-translation modification enzymes.

Cells. The present invention further comprises cells in which a procollagen or collagen, either alone or in combination with one or more post translational enzymes, is expressed both as mRNA and as a protein. Preferably, the procollagen or collagen (types I-XIX), and/or the post-translational enzyme, is expressed in mammalian cells, insect cells, or yeast cells. Notwithstanding these preferred embodiments, other cells, including plant cells and algae, can be manufactured.

In preferred embodiments of the present invention, cells such as mammalian, insect and yeast cells, which may not naturally produce sufficient amounts of post-translational enzymes, are transformed with at least one set of genes coding for a post-translational enzyme, such as prolyl 4-hydroxylase, C-proteinase, N-proteinase, lysyl oxidase or lysyl hydroxylase.

Polypeptides. The invention comprises the recombinant polypeptides expressed according to the methods of the present invention, including fusion products produced from chimeric genes wherein, for example, relevant epitopes of collagen or procollagen can be manufactured for therapeutic and other uses. The polypeptides of the present invention further include deglycosolated, unglycosolated and partially glycosolated collagens and procollagens.

An advantage of human recombinant collagens of the present invention is that these collagens will not produce allergic responses in man. Moreover, collagen of the present invention prepared from cultured cells should be of a higher quality than collagen obtained from animal sources, and should form larger and more tightly packed proteins. These higher quality proteins should form deposits in tissues that last much longer than the currently available commercial materials.

### BRIEF DESCRIPTION OF THE DRAWINGS

FIG. 1 is a photograph showing analysis by polyacrylamide gel electrophoresis in SDS of the proteins secreted into medium by HT-1080 cells that were transfected with a gene construct containing the promoter, first exon and most of the first intron of the human COL1A1 gene linked to 30 kb fragment containing all of COL2A1 except the first two exons.

FIG. 2 is a photograph evidencing the secretion type II procollagen into the medium from cells described in FIG. 1 was folded into a correct native conformation.

FIG. 3 is a photograph showing analysis of medium of HT-1080 cells co-transfected with a gene for COL1A1 and a gene for COL1A2.

FIG. 4 is a schematic representation of the cDNA for the proα1(I) chain of human type I procollagen that has been modified to contain artificial sites for cleavage by specific restriction endonucleases.

FIG. 5 is a photograph showing analysis by nondenaturing 7.5% polyacrylamide gel electrophoresis (lanes 1-3) and 10% polyacrylamide gel electrophoresis in SDS (lanes 4-6) of purified chick prolyl 4-hydroxylase (lanes 1 and 4) and the proteins secreted into medium by Sf9 cells expressing the gene for the a-subunit and the B-subunit of human prolyl 4-hydroxylase and infected with a58/B virus (lanes 2 and 5) or with a59/B virus (lanes 3 and 6). a58/B and a59/B differ by a stretch of 64 base pairs.

FIG. 6 is a gel showing the expression of recombinant human type III procollagen in Sf9 and High Five cells.

FIG. 7 is a gel showing the expression of recombinant human type I procollagen in insect cells, analyzed on a silver stained, 5% SDS-PAGE gel. Lane 1 is a pepsin digested sample from cells expressing only the proα1 chain of type I procollagen. Lane 2 is a pepsin digested sample from cells coexpressing proα1 and proα2 chains of type I procollagen.

FIG. 8 is a gel showing the expression of recombinant human type II procollagen in insect cells, analyzed on a coomassie stained 5% SDS-PAGE gel.

FIG. 9 is an SDS-PAGE analysis under reducing and nonreducing conditions of purified type III collagen. The gel was stained with Coomassie Brilliant Blue. The reduced type III collagen sample is shown in lane 2 and the nonreduced sample in lane 3. Molecular weight markers were run in lane 1. The positions of the trimeric α1 (III) chains and the monomeric α1 (III) chains are shown by arrows.

FIG. 10 is a non-reducing SDS-PAGE analysis of trimer formation of the proα1 (III) chains expressed in High Five insect cells. The samples were electrophoresed on 5% SDS-PAGE under nonreducing conditions and analyzed by Coomassie staining. Lane 1, molecular weight markers; lane 2, cell extract; lane 3, cell extract digested with pepsin; lane 4, proteins soluble in 1% SDS. The positions of the trimeric proα1 (III) and α1 (III) chains are shown by arrows.

FIG. 11 is an analysis of the thermal stability of the recombinant human type III collagen produced in insect cells by a brief protease digestion.

### DETAILED DESCRIPTION OF THE INVENTION

Definitions

The term “collagen” refers to any one of the collagen types I-XIX, as well as any novel collagens produced according to the methods of this invention. The term also encompasses both procollagen and mature collagen assembled as hetero- and homo-trimers, and any single chain polypeptides of procollagen or collagen for any of the collagen types, and any heterotrimers of any combination of the collagen constructs of the invention. The term “collagen” is meant to encompasses all of the foregoing, unless the context dictates otherwise.

The term “procollagen” refers to any one of the collagen types I-XIX, as well as any novel collagens produced by this invention, that possess additional C-terminal and/or N-terminal peptides that assist in trimer assembly, solubility, purification or other function, and then are subsequently cleaved by N-proteinase, C-proteinase or other proteins.

The term “collagen subunit” refers to the amino acid sequence of one polypeptide chain of a collagen protein encoded by a single gene, as well as derivatives, including deletion derivatives, conservative substitutions, etc.

A “fusion protein” is a protein in which peptide sequences from different proteins are covalently linked together.

The term “collagen post-translational enzyme” refers to any enzyme that modifies a procollagen, collagen, or components comprising a collagen molecule, and encompasses, but is not limited to, prolyl-4-hydroxylase, C-proteinase, N-proteinase, lysyl hydroxylase, and lysyl oxidase. The term “collagen post-translational enzyme” is meant to encompass all of the foregoing, unless the context dictates otherwise.

The term “infection” refers to the introduction of nucleic acids into an organism by use of a virus or viral vector, and preferably, baculovirus or Semliki Forest virus.

The term “transformation” means introducing DNA into an organism so that the DNA is replicable, either as an extrachromosomal element, or by chromosomal integration.

The term “transfection” refers to the taking up of an expression vector by a host cell, whether or not any coding sequences are in fact expressed.

The phrase “stringent conditions” as used herein refers to those hybridizing conditions that (1) employ low ionic strength and high temperature for washing, for example, 0.015 M NaCl/0.0015 M sodium citrate/0.1% SDS at 50° C.; or (2) employ during hybridization a denaturing agent such as formamide, for example, 50% (vol/vol) formamide with 0.1% bovine serum albumin/0.1% Ficoll/0.1l% polyvinylpyrrolidone/50 mM sodium phosphate buffer at pH 6.5 with 750 mM NaCl, 75 mM sodium citrate at 42° C.; or (3) employ 50% formamide, 5×SSC (0.75 M NaCl, 0.075 M Sodium citrate), 5×Denhardt's solution, sonicated salmon sperm DNA (50 g/ml), 0.1% SDS, and 10% dextran sulfate at 42° C., with washes at 42° C. in 0.2×SSC and 0.1% SDS.

The term “purified” as used herein denotes that the indicated collagen or procollagen is present in the substantial absence of other biological macromolecules, e.g., polynucleotides, proteins, and the like. The term “purified” as used herein preferably means at least 95% by weight, more preferably at least 99.8% by weight, of the indicated biological macromolecules present (but water, buffers, and other small molecules, especially molecules having a molecular weight of less than 1000 daltons, can be present).

The term “isolated” as used herein refers to a protein molecule separated not only from other proteins that are present in the natural source of the protein, but also from other proteins, and preferably refers to a protein found in the presence of (if anything) only a solvent, buffer, ion, or other component normally present in a solution of the same. The terms “isolated” and “purified” do not encompass proteins present in their natural source.

Nucleic Acids Related to the Present Invention

In accordance with the invention, polynucleotide sequences which encode any collagen subunit, or functional equivalents thereof, may be used to generate recombinant DNA molecules that direct the expression of that subunit of collagen, or a functional equivalent thereof, in appropriate host cells. Preferred embodiments of the invention are the polynucleotide sequences of collagen subunits of type I-type IV, type XIII, type XV, and type XVIII, or functional equivalents thereof.

The nucleic acid sequences encoding the known collagen types have been generally described in the art. See, e.g., Fukai et al., Methods of Enzymology 245:3-28 (1994) and references cited therein. New collagens/procollagens or known collagens/procollagens from which nucleic acid sequence is not available may be obtained from cDNA libraries prepared from tissues believed to possess a “novel” type of collagen and to express the novel collagen at a detectable level. For example, a cDNA library could be constructed by obtaining polyadenylated mRNA from a cell line known to express the novel collagen, or a cDNA library previously made to the tissue/cell type could be used. The cDNA library is screened with appropriate nucleic acid probes, and/or the library is screened with suitable polyclonal or monoclonal antibodies that specifically recognize other collagens. Appropriate nucleic acid probes include oligonucleotide probes that encode known portions of the novel collagen from the same or different species. Other suitable probes include, without limitation, oligonucleotides, cDNAs, or fragments thereof that encode the same or similar gene, and/or homologous genomic DNAs or fragments thereof. Screening the cDNA or genomic library with the selected probe may be accomplished using standard procedures known to those in the art, such as those described in chapters 10-12 of Sambrook et al., Molecular Cloning: A Laboratory Manual. New York, Cold Spring Harbor Laboratory Press, 1989. Other means for identifying novel collagens involve known techniques of recombinant DNA technology, such as by direct expression cloning or using the polymerase chain reaction (PCR) as described in U.S. Pat. No. 4,683,195, issued Jul. 28, 1987, or in section 14 of Sambrook et al., Molecular Cloning: A Laboratory Manual. Second Edition, Cold Spring Harbor Laboratory Press, New York, 1989, or in Chapter 15 of Current Protocols in Molecular Biology, Ausubel et al. eds., Greene Publishing Associates and Wiley-Interscience 1991.

Altered DNA sequences which may be used in accordance with the invention include deletions, additions or substitutions of different nucleotide residues resulting in a sequence that encodes the same or a functionally equivalent gene product. The gene product itself may contain deletions, additions or substitutions of amino acid residues within a collagen sequence, which result in a functionally equivalent collagen.

The nucleic acid sequences of the invention may be engineered in order to alter the collagen coding sequence for a variety of ends including, but not limited to, alterations which modify processing and expression of the gene product. For example, alternative secretory signals may be substituted for the native human secretory signal and/or mutations may be introduced using techniques which are well known in the art, e.g., site-directed mutagenesis, to insert new restriction sites, to alter glycosylation patterns, phosphorylation, etc. Additionally, when expressing in non-human cells, the polynucleotides encoding the collagens of the invention may be modified in the silent position of any triplet amino acid codon so as to better conform to the codon preference of the particular host organism.

The nucleic acid sequences of the invention are further directed to sequences which encode variants of the described collagens and fragments. These amino acid sequence variants of native collagens and collagen fragments may be prepared by methods known in the art by introducing appropriate nucleotide changes into a native or variant collagen encoding polynucleotide. There are two variables in the construction of amino acid sequence variants: the location of the mutation and the nature of the mutation. The amino acid sequence variants of collagen are preferably constructed by mutating the polynucleotide to give an amino acid sequence that does not occur in nature. These amino acid alterations can be made at sites that differ in collagens from different species (variable positions) or in highly conserved regions (constant regions). Sites at such locations will typically be modified in series, e.g., by substituting first with conservative choices (e.g., hydrophobic amino acid to a different hydrophobic amino acid) and then with more distant choices (e.g., hydrophobic amino acid to a charged amino acid), and then deletions or insertions may be made at the target site.

Amino acids are divided into groups based on the properties of their side chains (polarity, charge, solubility, hydrophobicity, hydrophilicity, and/or the amphipatic nature): (1) hydrophobic (leu, met, ala, ile), (2) neutral hydrophobic (cys, ser, thr), (3) acidic (asp, glu), (4) weakly basic (asn, gln, his), (5) strongly basic (lys, arg), (6) residues that influence chain orientation (gly, pro), and (7) aromatic (trp, tyr, phe). Conservative changes encompass variants of an amino acid position that are within the same group as the “native” amino acid. Moderately conservative changes encompass variants of an amino acid position that are in a group that is closely related to the “native” amino acid (e.g., neutral hydrophobic to weakly basic). Non-conservative changes encompass variants of an amino acid position that are in a group that is distantly related to the “native” amino acid (e.g., hydrophobic to strongly basic or acidic).

Amino acid sequence deletions generally range from about 1 to 30 residues, preferably about 1 to 10 residues, and are typically contiguous. Amino acid insertions include amino- and/or carboxyl-terminal fusions ranging in length from one to one hundred or more residues, as well as intrasequence insertions of single or multiple amino acid residues. Intrasequence insertions may range generally from about 1 to 10 amino residues, preferably from 1 to 5 residues. Examples of terminal insertions include the heterologous signal sequences necessary for secretion or for intracellular targeting in different host cells.

In a preferred method, polynucleotides encoding a collagen are changed via site-directed mutagenesis. This method uses oligonucleotide sequences that encode the polynucleotide sequence of the desired amino acid variant, as well as a sufficient adjacent nucleotide on both sides of the changed amino acid to form a stable duplex on either side of the site of being changed. In general, the techniques of site-directed mutagenesis are well known to those of skill in the art and this technique is exemplified by publications such as, Edelman et al., DNA 2:183 (1983). A versatile and efficient method for producing site-specific changes in a polynucleotide sequence was published by Zoller and Smith, Nucleic Acids Res. 10:6487-6500 (1982).

PCR may also be used to create amino acid sequence variants of a collagen. When small amounts of template DNA are used as starting material, primer(s) that differs slightly in sequence from the corresponding region in the template DNA can generate the desired amino acid variant. PCR amplification results in a population of product DNA fragments that differ from the polynucleotide template encoding the collagen at the position specified by the primer. The product DNA fragments replace the corresponding region in the plasmid and this gives the desired amino acid variant.

A further technique for generating amino acid variants is the cassette mutagenesis technique described in Wells et al., Gene 34:315 (1985); and other mutagenesis techniques well known in the art, such as, for example, the techniques in Sambrook et al., supra, and Current Protocols in Molecular Biology, Ausubel et al., supra.

In another embodiment of the invention, a collagen sequence may be ligated to a heterologous sequence to encode a fusion protein. For example, a fusion protein may be engineered to contain a cleavage site located between an α3(IX) collagen sequence and the heterologous protein sequence, so that the α3(IX) collagen may be cleaved away from the heterologous moiety.

Due to the inherent degeneracy of the genetic code, other DNA sequences which encode substantially the same or a functionally equivalent amino acid sequence may be used in the practice of the invention for the cloning and expression of these collagen proteins. Such DNA sequences include those which are capable of hybridizing to the appropriate human collagen sequence under stringent conditions.

Collagen Modifying Polypeptides and Corresponding Nucleic Acid Sequences

As naturally produced, collagens are structural proteins comprised of one or more collagen subunits which together form at least one triple-helical domain. A variety of enzymes are utilized in order to transform the collagen subunits into procollagen or other precursor molecules and then mature collagen. Such enzymes include prolyl-4-hydroxylase, C-proteinase, N-proteinase, lysyl oxidase and lysye hydroxylase.

Prolyl 4-hydroxylase plays a central role in the biosynthesis of all collagens, as the 4-hydroxyproline residues stabilize the folding of the newly synthesized polypeptide chains, into triple-helical molecules. Prockop et al., Annu. Rev. Biochem. 64:403-434 (1995); Kivirikko et al., “Post-Translational Modifications of Proteins,” pp. 1-51 (1992); Kivirikko et al., FASEB J. 3:1609-1617 (1989). For example, when the proα1 chains of type III procollagen were expressed in insect cells, without recombinant prolyl 4-hydroxylase, considerable amounts of procollagen were made in the cells, and the proα1 chains formed triple-helical molecules as indicated by the resistance of the collagenous domains of the collagen to protease degradation at 22° C. However, the Tₘ of the triple helices of such molecules was about 6° C. lower than procollagen produced in the presence of the recombinant prolyl 4-hydroxylase. Also, the level of expression of type III collagen was lower in the absence of recombinant prolyl 4-hydroxylase than in its presence.

Lysyl hydroxylase, an α2 homodimer, catalyzes the post-translation modification of collagen to form hydroxylysine in collagens. See generally, Kivirikko et al., Post-Translational Modifications of Proteins, Harding, J. J., and Crabbe, M. J. C., eds., CRC Press, Boca Raton, Fla. (1992); Kivirikko, Principles of Medical Biology, Vol. 3 Cellular Organelles and the Extracellular Matrix, Bittar, E. E., and Bittar, N., eds., JAI Press, Greenwich, Great Britain (1995).

C-proteinase processes the assembled procollagen by cleaving off the C-terminal ends of the procollagens that assist in assembly of, but are not part of, the triple helix of the collagen molecule. See generally, Kadler et al., J. Biol. Chem. 262:15969-15701 (1987), Kadler et al., Ann. NY Acad. Sci. 580:214-224 (1990).

N-proteinase processes the assembled procollagen by cleaving off the N-terminal ends of the procollagens that assist in the assembly of, but are not part of, the collagen triple helix. See generally, Hojima et al., J. Biol. Chem. 269:11381-11390 (1994).

Lysyl oxidase is an extracellular copper enzyme that catalyzes the oxidative deamination of the ε-amino group in certain lysine and hydroxy lysine residues to form a reactive aldehyde. These aldehydes then undergo an aldol condensation to form aldols, which cross links collagen fibrils. Information on the DNA and protein sequence of lysyl oxidase can found, among elsewhere, in Kivirikko, Principles of Medical Biology, Vol. 3 Cellular Organelles and the Extracellular Matrix, Bittar, E. E., and Bittar, N., eds., JAI Press, Greenwich, Great Britain (1995), Kagan, Path. Res. Pract. 190: 910-919 (1994), Kenyon et al., J. Biol. Chem. 268(25):18435-18437 (1993), Wu et al., J. Biol. Chem. 267(34):24199-24206 (1992), Mariani et al., Matrix 12(3):242-248 (1992), and Hamalainen et al., Genomics 11(3):508-516 (1991).

The nucleic acid sequences encoding a number of these post-translational enzymes have been reported. See e.g. Vuori et al., Proc. Natl. Acad. Sci. USA 89:7467-7470 (1992); Kessler et al., Science 271:360-362 (1996). The nucleic acid sequences encoding the various post-translational enzymes may also be determined according to the methods generally described above and include use of appropriate probes and nucleic acid libraries.

Host-Vector Systems for Expressing Recombinant Collagen

In order to express the collagens and related collagen post-translational enzymes of the invention, the nucleotide sequence encoding the collagen, or a functional equivalent, is inserted into an appropriate expression vector, i.e., a vector which contains the necessary elements for the transcription and translation of the inserted coding sequence, or in the case of an RNA viral vector, the necessary elements for replication and translation.

Methods which are well known to those skilled in the art can be used to construct expression vectors containing a collagen coding sequence for the collagens of the invention and appropriate transcriptional/translational control signals. These methods include in vitro recombinant DNA techniques, synthetic techniques and in vivo recombination/genetic recombination. see, for example, the techniques described in Maniatis et al., Molecular Cloning A Laboratory Manual, Cold Spring Harbor Laboratory, N.Y. (1989) and Ausubel et al., Current Protocols in Molecular Biology, Greene Publishing Associates and Wiley Interscience, N.Y. (1989).

A variety of host-expression vector systems may be utilized to express a collagen coding sequence. These include, but are not limited to, microorganisms such as bacteria transformed with recombinant bacteriophage DNA, plasmid DNA or cosmid DNA expression vectors containing a procollagen or collagen coding sequence; yeast or filamentous fungi transformed with recombinant yeast or fungi expression vectors containing a procollagen or collagen coding sequence; insect cell systems infected with recombinant virus expression vectors (e.g., baculovirus) containing sequence encoding the procollagen or collagen of the invention; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a procollagen or collagen coding sequence; or animal cell systems. The expression elements of these systems vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter) and the like may be used; when cloning in insect cell systems, promoters such as the baculovirus polyhedron promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of a collagen DNA, SV40-, BPV- and EBV-based vectors may be used with an appropriate selectable marker.

In bacterial systems a number of expression vectors may be advantageously selected depending upon the use intended for the collagen expressed. For example, when large quantities of the collagens of the invention are to be produced for the generation of antibodies, vectors which direct the expression of high levels of fusion protein products that are readily purified may be desirable. Such vectors include, but are not limited to, the E. coli expression vector pUR278 (Ruther et al., EMBO J. 2:1791 (1983)), in which the collagen coding sequence may be ligated into the vector in frame with the lac Z coding region so that a hybrid AS-lac Z protein is produced; pIN vectors (Inouye et al., Nucleic Acids Res. 13:3101-3109 (1985); Van Heeke et al., J. Biol. Chem. 264:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned polypeptide of interest can be released from the GST moiety.

A preferred expression system is a yeast expression system. In yeast, a number of vectors containing constitutive or inducible promoters may be used. For a review see, Current Protocols in Molecular Biology, Vol. 2, Ed. Ausubel et al., Greene Publish. Assoc. & Wiley Interscience, Ch. 13 (1988); Grant et al., Expression and Secretion Vectors for Yeast, in Methods in Enzymology, Ed. Wu & Grossman, Acad. Press, N.Y. 153:516-544 (1987); Glover, DNA Cloning, Vol. II, IRL Press, Wash., D.C., Ch. 3 (1986); Bitter, Heterologous Gene Expression in Yeast, in Methods in Enzymology, Eds. Berger & Kimmel, Acad. Press, N.Y. 152:673-684 (1987); and The Molecular Biology of the Yeast Saccharomyces, Eds. Strathern et al., Cold Spring Harbor Press, Vols. I and II (1982).

A particularly preferred system useful for cloning and expression of the collagen proteins of the invention uses host cells from the yeast Pichia. Species of non-Saccharomyces yeast such as Pichia pastoris appear to have special advantages in producing high yields of recombinant protein in scaled up procedures. Additionally, a Pichia expression kit is available from Invitrogen Corporation (San Diego, Calif.).

There are a number of methanol responsive genes in methylotrophic yeasts such as Pichia pastoris, the expression of each being controlled by methanol responsive regulatory regions (also referred to as promoters). Any of such methanol responsive promoters are suitable for use in the practice of the present invention. Examples of specific regulatory regions include the promoter for the primary alcohol oxidase gene from Pichia pastoris AOX1, the promoter for the secondary alcohol oxidase gene from P. pastoris AXO2, the promoter for the dihydroxyacetone synthase gene from P. pastoris (DAS), the promoter for the P40 gene from P. pastoris, the promoter for the catalase gene from P. pastoris, and the like.

Typical expression in Pichia pastoris is obtained by the promoter from the tightly regulated AOX1 gene. See Ellis et al., Mol. Cell. Biol. 5:1111 (1985) and U.S. Pat. No. 4,855,231. This promoter can be induced to produce high levels of recombinant protein after addition of methanol to the culture. By subsequent manipulations of the same cells, expression of genes for the collagens of the invention described herein is achieved under conditions where the recombinant protein is adequately hydroxylated by prolyl 4-hydroxylase and, therefore, can fold into a stable helix that is required for the normal biological function of the proteins in forming fibrils.

Another particularly preferred yeast expression system makes use of the methylotrophic yeast Hansenula polymorpha. Growth on methanol results in the induction of key enzymes of the methanol metabolism, namely MOX (methanol oxidase), DAS (dihydroxyacetone synthase) and FMHD (formate dehydrogenase). These enzymes can constitute up to 30-40% of the total cell protein. The genes encoding MOX, DAS, and FMDH production are controlled by very strong promoters which are induced by growth on methanol and repressed by growth on glucose. Any or all three of these promoters may be used to obtain high level expression of heterologous genes in H. polymorpha. The gene encoding a collagen of the invention is cloned into an expression vector under the control of an inducible H. polymorpha promoter. If secretion of the product is desired, a polynucleotide encoding a signal sequence for secretion in yeast, such as the S. cerevisiae prepro-mating factor α1, is fused in frame with the coding sequence for the collagen of the invention. The expression vector preferably contains an auxotrophic marker gene, such as URA3 or LEU2, which may be used to complement the deficiency of an auxotrophic host.

The expression vector is then used to transform H. polymorpha host cells using techniques known to those of skill in the art. An interesting and useful feature of H. polymorpha transformation is the spontaneous integration of up to 100 copies of the expression vector into the genome. In most cases, the integrated DNA forms multimers exhibiting a head-to-tail arrangement. The integrated foreign DNA has been shown to be mitotically stable in several recombinant strains, even under non-selective conditions. This phenomena of high copy integration further adds to the high productivity potential of the system.

Filamentous fungi may also be used to produce the collagens of the instant invention. Vectors for expressing and/or secreting recombinant proteins in filamentous fungi are well known, and one of skill in the art could use these vectors to express recombinant collagen.

In cases where plant expression vectors are used, the expression of sequences encoding the collagens of the invention may be driven by any of a number of promoters. For example, viral promoters such as the 35S RNA and 19S RNA promoters of CaMV (Brisson et al., Nature 310:511-514 (1984), or the coat protein promoter of TMV (Takamatsu et al., EMBO J. 6:307-311 (1987)) may be used; alternatively, plant promoters such as the small subunit of RUBISCO (Coruzzi et al., EMBO J. 3:1671-1680 (1984); Broglie et al., Science 224:838-843 (1984); or heat shock promoters, e.g., soybean hsp17.5-E or hsp17.3-B (Gurley et al., Mol. Cell. Biol. 6:559-565 (1986) may be used. These constructs can be introduced into plant cells using Ti plasmids, Ri plasmids, plant virus vectors, direct DNA transformation, microinjection, electroporation, etc. For reviews of such techniques see, for example, Weissbach & Weissbach, Methods for Plant Molecular Biology, Academic Press, NY, Section VIII, pp. 421-463 (1988); and Grierson & Corey, Plant Molecular Biology, 2d Ed., Blackie, London, Ch. 7-9 (1988).

An alternative expression system which could be used to express the collagens of the invention is an insect system. In one such system, Autographa californica nuclear polyhidrosis virus (AcNPV) is used as a vector to express foreign genes. The virus grows in Spodoptera frugiperda cells. Coding sequence for the collagens of the invention may be cloned into non-essential regions (for example the polyhedron gene) of the virus and placed under control of an AcNPV promoter (for example, the polyhedron promoter). Successful insertion of a collagen coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed. (see, e.g., Smith et al., J. Virol. 46:584 (1983); Smith, U.S. Pat. No. 4,215,051). Further examples of this expression system may be found in Current Protocols in Molecular Biology, Vol. 2, Ed. Ausubel et al., Greene Publish. Assoc. & Wiley Interscience.

In mammalian host cells, a number of viral based expression systems may be utilized. In cases where an adenovirus is used as an expression vector, coding sequence for the collagens of the invention may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing collagen in infected hosts. (See, e.g., Logan & Shenk, Proc. Natl. Acad. Sci. USA 81:3655-3659 (1984)). Alternatively, the vaccinia 7.5K promoter may be used. (See, e.g., Mackett et al., Proc. Natl. Acad. Sci. USA 79:7415-7419 (1982); Mackett et al., J. Virol. 49:857-864 (1984); Panicali et al., Proc. Natl. Acad. Sci. USA 79:4927-4931 (1982).

Specific initiation signals may also be required for efficient translation of inserted collagen coding sequences. These signals include the ATG initiation codon and adjacent sequences. In cases where the entire collagen gene, including its own initiation codon and adjacent sequences, is inserted into the appropriate expression vector, no additional translational control signals may be needed. However, in cases where only a portion of a collagen coding sequence is inserted, exogenous translational control signals, including the ATG initiation codon, must be provided. Furthermore, the initiation codon must be in phase with the reading frame of the collagen coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:516-544 (1987)).

Preferably, the collagens of the invention are expressed as secreted proteins. When the engineered cells used for expression of the proteins are non-human host cells, it is often advantageous to replace the human secretory signal peptide of the collagen protein with an alternative secretory signal peptide which is more efficiently recognized by the host cell's secretory targeting machinery. The appropriate secretory signal sequence is particularly important in obtaining optimal fungal expression of mammalian genes. For example, in methylotrophic yeasts, a DNA sequence encoding the in-reading frame S. cerevisiae α-mating factor pre-pro sequence may be inserted at the amino-terminal end of the coding sequence. The αMF pre-pro sequence is a leader sequence contained in the αMF precursor molecule, and includes the lys-arg encoding sequence which is necessary for proteolytic processing and secretion (see, e.g., Brake et al., Proc. Natl. Acad. Sci. USA 81:4642 (1984)). Other signal sequences for prokaryotic, yeast, fungi, insect or mammalian cells are well known in the art, and one of ordinary skill could easily select a signal sequence appropriate for the host cell of choice.

The vectors of this invention may autonomously replicate in the host cell, or may integrate into the host chromosome. Suitable vectors with autonomously replicating sequences (“ars”) are well known for a variety of bacteria (e.g., the ars from pBR322 functions in the majority of gram negative bacteria), yeast (the 2μ plasmid ars), and various viral replications sequences for both prokaryotes and eukaryotes (prokaryote: λ, T-even phages, M13, etc; eukaryote: adenovirus, SV40, polyoma, VSV or BPV, vaccina, etc.). Vectors may integrate into the host cell genome when they have a DNA sequence that is homologous to a sequence found in the host cell's genomic DNA.

The vectors of the invention also encode a selection gene, also termed a selectable marker, that encodes a product necessary for the host cell to grow and survive under certain conditions. Typical selection genes include genes encoding (1) a protein that confers resistance to an antibiotic or other toxin (e.g., tetracycline, ampicillin, neomycin, methotrexate, etc.), and (2) a protein that complements an auxotrophic requirement of the host cell, etc. Other examples of selection genes include: the herpes simplex virus thymidine kinase (Wigler et al., Cell 11:223 (1977)), hypoxanthine-guanine phosphoribosyltransferase (Szybalska et al., Proc. Natl. Acad. Sci. USA 48:2026 (1962)), and adenine phosphoribosyltransferase (Lowy et al., Cell 22:817-(1980)) genes that can be employed in tk, hgprt or aprt cells, respectively. Also, antimetabolite resistance can be used as the basis of selection for dhfr, which confers resistance to methotrexate (Wigler et al., Natl. Acad. Sci. USA 77:3567 (1980); O'Hare et al., Proc. Natl. Acad. Sci. USA 78:1527 (1981)); gpt, which confers resistance to mycophenolic acid (Mulligan et al., Proc. Natl. Acad. Sci. USA 78:2072 (1981)); neo, which confers resistance to the aminoglycoside G-418 (Colberre-Garapin et al., J. Mol. Biol. 150:1 (1981)); and hygro, which confers resistance to hygromycin (Santerre et al., Gene 30:147 (1984)). Recently, additional selectable genes have been described, namely trpB, which allows cells to utilize indole in place of tryptophan; hisD, which allows cells to utilize histinol in place of histidine (Hartman et al., Proc. Natl. Acad. Sci. USA 85:8047 (1988)); and ODC (ornithine decarboxylase) which confers resistance to the ornithine decarboxylase inhibitor, 2-(difluoromethyl)-DL-ornithine, DFMO (McConlogue L., In: Current Communications in Molecular Biology, Cold Spring Harbor Laboratory, Ed. (1987)).

Further regulatory elements necessary for the expression vectors of the invention include sequences for initiating transcription, e.g., promoters and enhancers. Promoters are untranslated sequences located upstream from the start codon of the structural gene that control the transcription of the nucleic acid under its control. Inducible promoters are promoters that alter their level of transcription initiation in response to a change in culture conditions, e.g., the presence or absence of a nutrient. One of skill in the art would know of a large number of promoters that would be recognized in host cells suitable for the present invention. These promoters are operably linked to the DNA encoding the collagen by removing the promoter from its native gene and placing the collagen encoding DNA 3′ of the promoter sequence. Promoters useful in the present invention include, but are not limited to, the following: (prokaryote) (1) the lactose promoter, the alkaline phosphatase promoter, the tryptophan promoter, and hybrid promoters such as the tac promoter, (yeast) (2) the promoter for 3-phosphoglycerate kinase, other glycolytic enzyme promoters (hexokinase, pyruvate decarboxylase, phophofructosekinase, glucose-6-phosphate isomerase, etc.), the promoter for alcohol dehydrogenase, the metallothionein promoter, the maltose promoter, and the galactose promoter, (eukaryotic) (3) virtually all eukaryotic genes have an AT-rich region located approximately 25 to 30 bases upstream from the site where transcription is initiated, examples of suitable eukaryotic promoters include: promoters from the viruses polyoma, fowlpox, adenovirus, bovine papilloma virus, avian sarcoma virus, cytomegalovirus, retroviruses, SV40, and promoters from the target eukaryote including: the glucoamylase promoter from Aspergillus, the actin promoter or an immunoglobin promoter from a mammal, and native collagen promoters. See, e.g., de Boer et al., Proc. Natl. Acad. Sci. USA 80:21-25 (1983), Hitzeman et al., J. Biol. Chem. 255:2073 (1980), Fiers et al., Nature 273:113 (1978), Mulligan and Berg, Science 209:1422-1427 (1980), Pavlakis et al., Proc. Natl. Acad. Sci. USA 78:7398-7402 (1981), Greenway et al., Gene 18:355-360 (1982), Gray et al., Nature 295:503-508 (1982), Reyes et al., Nature 297:598-601 (1982), Canaani and Berg, Proc. Natl. Acad. Sci. USA 79:5166-5170 (1982), Gorman et al., Proc. Natl. Acad. Sci. USA 79:6777-6781 (1982), Nunberg et al., Mol. and Cell. Biol. 11(4):2306-2315 (1984).

Transcription of the collagen encoding DNA from the promoter is often increased by inserting an enhancer sequence in the vector. Enhancers are cis-acting elements, usually about from 10 to 300 bp, that act to increase the rate of transcription initiation at a promoter. Many enhancers are known for both eukaryotes and prokaryotes, and one of ordinary skill could select an appropriate enhancer for the host cell of interest. See, e.g., Yaniv, Nature 297:17-18 (1982) for eukaryotic enhancers.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cells lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include, but are not limited to, CHO, VERO, BHK, HeLa, COS, MDCK, 293, WI38, etc. Additionally, host cells may be engineered to express various enzymes to ensure the proper processing of the collagen molecules. For example, the gene for prolyl-4-hydroxylase may be coexpressed with the collagen gene in the host cell.

For long-term, high-yield production of recombinant proteins, stable expression is preferred. For example, cell lines which stably express the collagens of the invention may be engineered. Rather than using expression vectors which contain viral origins of replication, host cells can be transformed with collagen encoding DNA controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express a desired collagen.

Infection, Transformation and Transfection

Host cells are transfected or preferably infected or transformed with the above-described expression vectors, and cultured in nutrient media appropriate for selecting transductants or transformants containing the collagen encoding vector.

The host cells which contain the coding sequence and which express the biologically active gene product may be identified by at least four general approaches; (a) DNA-DNA or DNA-RNA hybridization; (b) the presence or absence of “marker” gene functions; (c) assessing the level of transcription as measured by the expression of collagen mRNA transcripts in the host cell; and (d) detection of the gene product as measured by immunoassay or by its biological activity.

In the first approach, the presence of the collagen coding sequence inserted in the expression vector can be detected by DNA-DNA or DNA-RNA hybridization using probes comprising nucleotide sequences that are homologous to the collagen coding sequence, respectively, or portions or derivatives thereof.

In the second approach, the recombinant expression vector/host system can be identified and selected based upon the presence or absence of certain “marker” gene functions (e.g., thymidine kinase activity, resistance to antibiotics, resistance to methotrexate, transformation phenotype, occlusion body formation in baculovirus, etc.). For example, if the collagen coding sequence is inserted within a marker gene sequence of the vector, recombinant cells containing collagen coding sequence can be identified by the absence of the marker gene function. Alternatively, a marker gene can be placed in tandem with the collagen sequence under the control of the same or different promoter used to control the expression of the collagen coding sequence. Expression of the marker in response to induction or selection indicates expression of the collagen coding sequence.

In the third approach, transcriptional activity of the collagen coding region can be assessed by hybridization assays. For example, RNA can be isolated and analyzed by Northern blot using a probe homologous to the collagen coding sequence or particular portions thereof. Alternatively, total nucleic acids of the host cell may be extracted and assayed for hybridization to such probes.

In the fourth approach, the expression of a collagen protein product can be assessed immunologically, for example by Western blots, immunoassays such as radioimmuno-precipitation, enzyme-linked immunoassays and the like.

Purification of Collagens

The expressed collagen of the invention, which is preferably secreted into the culture medium, is purified to homogeneity by chromatography. In one embodiment, the recombinant collagen protein is purified by size exclusion chromatography. However, other purification techniques known in the art can also be used, including ion exchange chromatography, and reverse-phase chromatography. See, e.g., Maniatis et al., Molecular Cloning A Laboratory Manual, Cold Spring Harbor Laboratory, N.Y. (1989), Ausubel et al., Current Protocols in Molecular Biology, Greene Publishing Associates and Wiley Interscience, N.Y. (1989), and Scopes, Protein Purification: Principles and Practice, Springer-Verlag New York, Inc., NY (1994).

The present invention is further illustrated by the following examples, which are not intended to be limited in any way.

### EXAMPLES

### Example 1

Synthesis of Human Type II Procollagen

A recombinant COL1A1 gene construct employed in the present invention comprised a fragment of the 5′-end of COL1A1 having a promotor, exon 1 and intron 1 fused to exons 3 through 54 of a COL2A1 gene. The hybrid construct was transfected into HT-1080 cells. These cells were co-transfected with a neomycin-resistance gene and grown in the presence of the neomycin analog G418. The hybrid construct was used to generate transfected cells.

A series of clones were obtained that synthesized mRNA for human type II procollagen. To analyze the synthesized proteins, the cells were incubated with [¹⁴C] proline so that the medium proteins could be analyzed by autoradiography (storage phosphor film analyzer).

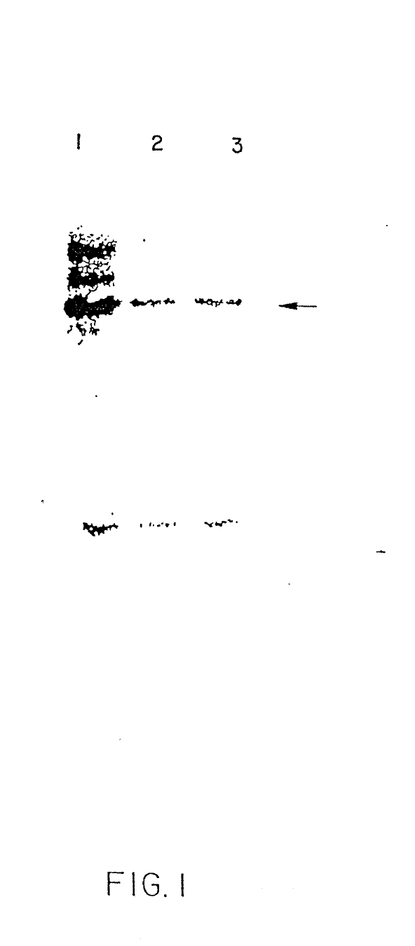

As set forth at FIG. 1, lane 1 shows that the unpurified medium proteins are comprised of three major polypeptide chains. Specifically, the medium proteins contained the expected type II procollagen comprised of proα1(II) chains together with proα1(IV) and proα2(IV) chains of type IV collagen normally synthesized by the cells. The upper two are proα1(IV) and proα2 (IV) chains of type IV collagen that are synthesized by cells not transfected by the construct. The third band is the proα1 (II) chains of human type II procollagen synthesized from the construct. Lanes 2 and 3 are the same medium protein after chromatography of the medium on an ion exchange column. As indicated in Lanes 2 and 3, the type II procollagen was readily purified by a single step of ion exchange chromatography.

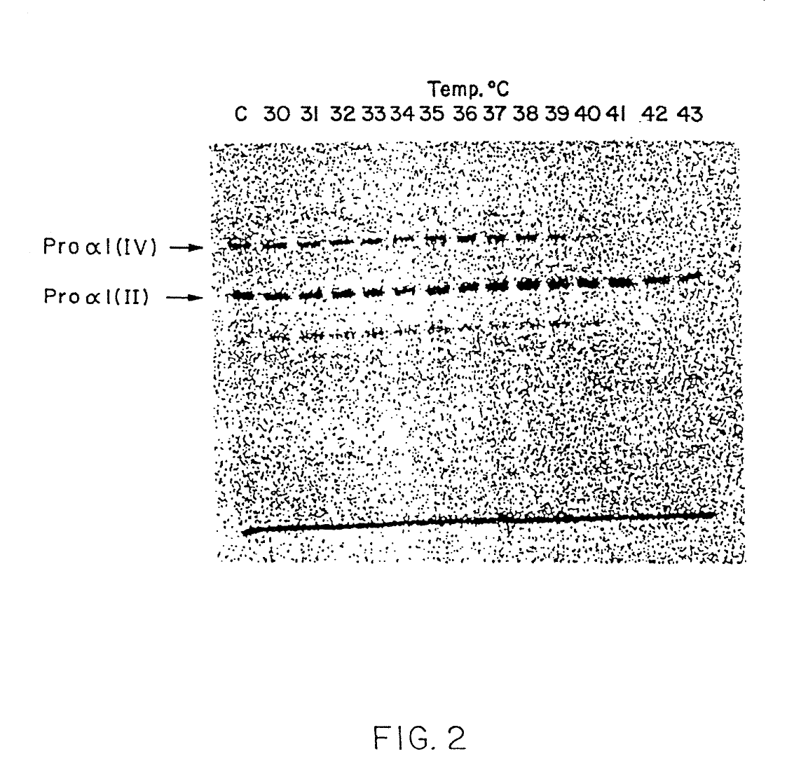

The type II procollagen secreted into the medium was correctly folded by a protease-thermal stability test. As evidenced at FIG. 2, the medium proteins were digested at the temperatures indicated with a high concentration of trypsin and chymotrypsin under conditions in which correctly folded triple-helical procollagen or collagen resists digestion but unfolded or incorrectly folded procollagen of collagen is digested to small fragments. The products of the digestion were than analyzed by polyacrylamide gel electrophoresis in SDS and fluorography. The results show that the type II procollagen resisted digestion up to 43° C., the normal temperature at which type II procollagen unfolds. Therefore, the type II procollagen is correctly folded and can be used to generate collagen fibrils.

### Example 2

Synthesis of Human Type I Procollagen

As a second example, HT-1080 cells were co-transfected with a COL1A1 gene and a COL1A2 gene. Both genes consisted of a cytomegalic virus promoter linked to a full-length cDNA. The COL1A2 gene construct but not the COL1A1 gene construct contained a neomycin-resistance gene. The cells were selected for expression of the COL1A2-neomycin resistance gene construct by growth in the presence of the neomycin-analog G418. The medium was then examined for expression of the COL1A1 with a specific polyclonal antibody for human proα1(1) chains.

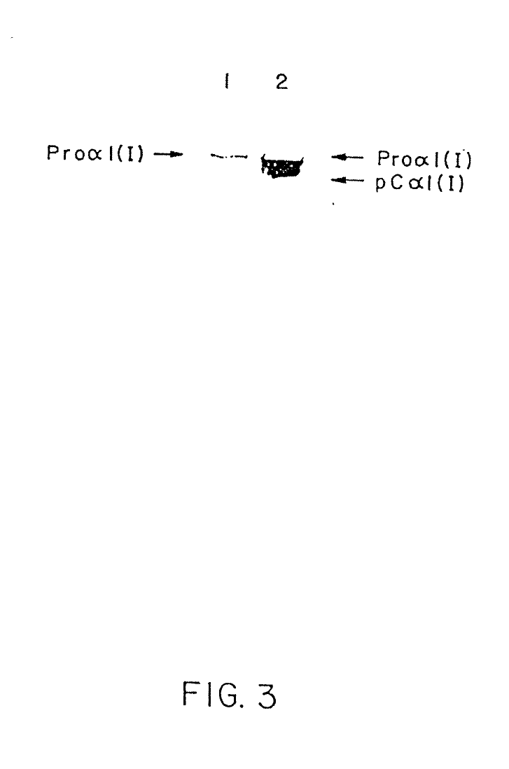

More specifically, the COL1A2 was linked to an active neomycin-resistance gene but the COL1A1 was not. The cells were screened for expression of the COL1A2-neomycin resistance gene construct with the neomycin analog G418. The medium was analyzed for expression of the COL1A1 by Western blotting with a polyclonal antibody specific for the human proα1(I) chain. As set forth in FIG. 3, lane 1 indicates that the medium proteins contained proα(I) chains (α1(I) and α2(I)). Lane 2 is an authentic standard of type I procollagen containing proα1(I) and proα2(I) chains and partially processed pCα1(I) chains. The results demonstrate that the cells synthesized human type procollagen that contained proα1(I) chains, presumably in the form of the normal heterotrimer with the composition two proα(I) chains and one proα2(I) chain.

These results demonstrated that the cells synthesized human type I procollagen that was probably comprised of the normal heterotrimeric structure of two proα1(I) chains and one proα2(I) chain.

Table 1 presents a summary of some of the DNA constructs containing human procollagen genes. The constructs were assembled from discrete fragments of the genes or cDNAs from the genes together with appropriate promoter fragments.

TABLE 1

| | | Central | | Protein |
|---|---|---|---|---|
| Constructs | 5′ end | Region | 3′ end | product |
| A | Promoter | Exons 3 | 3.5 kb | Human type |
| | (2.5 kb) + | to 54 | SphI/SphI | II |
| | exon 1 | from | fragment | procollagen, |
| | + intron 1 | COL2A1 | from | [proα1 (II)]³ |
| | from COL1A1 | | 3′ end of | |
| | | | COL2A1 | |
| B | Promoter | Exons 1 | 3.5 kb | Human type |
| | (2.5 kb) of | to 54 | SphI/SphI | II |
| | COL1A1 | from | fragment | procollagen |
| | | COL2A1 | from | [proα1 (II)] |
| | | | 3′ end of | |
| | | | COL2A1 | |
| C | Promoter | cDNA | 0.5 kb | Human type I |
| | (2.5 kb) + | for | fragment | procollagen, |
| | exon 1 + | COL1A1 | from | [proα1 (I)]₃ |
| | intron 1 + | except | COL1A1 | |
| | half of | for | | |
| | exon 2 from | first 1 | | |
| | COL1A1 | ½ | | |
| | | exons | | |
| D | Cytomegalic | cDNA | | Human type I |
| | virus | from | | procollagen, |
| | promoter | COL1A1 | | [proα1 (I)]₃ |
| E | Cytomegalic | cDNA | | Human type I |
| | virus | from | | [proα1 (I)]₂ |
| | promoter | COL1A2 | | proα2 (I)] |
| | | | | when |
| | | | | expressed |
| | | | | with |
| | | | | construct C |
| | | | | or D |

### Example 3

Cell Transfections

For cell transfection experiments, a cosmid plasmid clone containing the gene construct was cleaved with a restriction endonuclease to release the construct from the vector. A plasmid vector comprising a neomycin resistance gene, (Law et al., Mol. Cell. Biol. 3:2110-2115 (1983)) was linearized by cleavage with BamHI. The two samples were mixed in a ratio of approximately 10:1 gene construct to neomycin resistant gene, and the mixture was then used for cotransfection of HT-1080 cells by calcium phosphate coprecipitation (Sambrook et al., Molecular Cloning. A Laboratory Manual, Cold Spring Harbor Laboratory Press, 2d Edition (1989)). DNA in the calcium phosphate solution was layered onto cultured cells without 10 μg of chimeric gene construct per 100 ml plate of preconfluent cells. Cells were incubated in DMEM containing 10% newborn calf serum for 10 hours. The samples were subjected to glycerol shock by adding a 15% glycerol solution for 3 minutes. The cells were then transferred to DMEM medium containing newborn calf serum for 24 hours and then to the same medium containing 450 μg/ml of G418. Incubation in the medium containing G418 was continued for about 4 weeks with a change of medium every third day. G418-resistant cells were either pooled or separate clones obtained by isolating foci with a plastic cylinder and subcultured.

### Example 4

Western blotting

For assay of expression of the COL2A1 gene, polyclonal antibodies were prepared in rabbits using a 23-residue synthetic peptide that had an amino acid sequence found in the COOH-terminal telopeptide of type II collagen. See generally, Cheah et al., Proc. Natl. Acad. Sci. USA 82:2555-2559 (1985). The antibody did not react by Western blot analysis with proα chains of human type I procollagen or collagen, human type II procollagen or collagen, or murine type I procollagen. For assay of expression of the COL1A1 genes, polyclonal antibodies that reacted with the COOH-terminal polypeptide of the proα(I) chain were employed. See generally, Olsen et al., J. Biol. Chem. 266:1117-1121 (1991).

Culture medium from pooled clones or individual clones was removed and separately precipitated by the addition of solid ammonium sulfate to 30% saturation and precipitates were collected by centrifugation at 14,000×g and then dialyzed against a buffer containing 0.15 M NaCl, 0.5 mM EDTA, 0.5 mM N-ethylmaleimide, 0.1 mM and p-aminobenzamidine, and 50 mM Tris-HCl (pH 7.4 at 4° C.). Aliquots of the samples were heated to 10° C. for 5 minutes in 1% SDS, 50 mM DTT and 10% (v/v) glycerol, and separated by electrophoresis on 6% polyacrylamide gels using a mini-gel apparatus (Holford SE250, Holford Scientific) run at 125 V for 90 minutes. Separated proteins were electroblotted from the polyacrylamide gel at 40 V for 90 minutes onto a supported nitrocellulose membrane (Schleicher and Schuell). The transferred proteins were reacted for 30 minutes with the polyclonal antibodies at a 1:500 (v/v) dilution. Proteins reacting with the antibodies were detected with a secondary anti-rabbit IgG antibody coupled to alkaline phosphatase (Promega Biotech) for 30 minutes. Alkaline phosphatase was visualized with NBT/BCIP (Promega Biotech) as directed by the manufacturer.

### Example 5

In vitro Analysis of Recombinant Collagen

A. Assembly Of Recombinant Collagen: Protease Digestion.

To demonstrate that the procollagens synthesized and secreted in the medium by the transfected cells were correctly folded, the medium proteins were digested with high concentrations of proteases under conditions in which only correctly folded procollagens and collagens resist digestion. For digestion with a combination of trypsin and chymotrypsin, the cell layer from a 25 cm flask was scraped into 0.5 ml of modified Krebs II medium containing 10 mM EDTA and 0.1% Nonidet P-40 (Sigma). The cells were vigorously agitated in a Vortex mixer for 1 minute and immediately cooled to 4° C. The supernatant was transferred to new tubes. The sample was preincubated at the temperature indicated for 10 minutes and the digestion was carried out at the same temperature for 2 minutes. For the digestion, a 0.1 volume of the modified Krebs II medium containing 1 mg/ml trypsin and 2.5 mg/ml α-chymotrypsin (Boehringer Manheim) was added. The digestion was stopped by adding a 0.1 volume of 5 mg/ml soybean trypsin inhibitor (Sigma).

For analysis of the digestion products, the sample was rapidly immersed in boiling water for 2 minutes with the concomitant addition of a 0.2 volume of 5×electrophoresis sample buffer that consisted of 10% SDS, 50% glycerol, and 0.012% bromphenol blue in 0.625 M Tris-HCl buffer (pH 6.8). Samples were applied to SDS gels with prior reduction by incubating for 3 minutes in boiling water after the addition of 2% 2-mercaptoethanol. Electrophoresis was performed using the discontinuous system of Laemli, Nature 227:680-685 (1979), with minor modifications described by de Wet et al., J. Biol. Chem. 258:7721-7728 (1983).

B. Double Immunostaining of Sf9 Cells.

Sf9 cells were grown on glass slides and fixed in 100% ethanol at −20° C. Alternatively, cells in monolayer were detached, washed twice with a solution of 0.15 M NaCl and 0.02 M phosphate, pH 7.4 (washing solution), suspended in cold ethanol and spread on silanated (Maples, J. A., (1985), Am. J. Clin. Pathol. 83:356-363) glass slides. Cells were incubated with 1% bovine serum albumin in 0.15 M NaCl and 0.02 M phosphate, pH 7.4, for 15 min followed by incubation for 30 min in a 1:50 dilution of a mouse monoclonal antibody to the β subunit (5B5, Dako) and a rabbit polyclonal antibody to the α subunit of human prolyl 4-hydroxylase in the above bovine serum albumin-containing solution. Cells were washed with the washing solution 4 times for 20 min and incubated in a 1:10 dilution of a sheep anti-mouse Ig-rhodamine F(ab)2 fragment (Boehringer Mannheim) and a sheep anti-rabbit IgG fluorescein F(ab)2 fragment (Boehringer Mannheim) in the bovine serum albumin-containing solution for 30 min, washed with the washing solution, rinsed with distilled water and mounted using Glycergel (Dako). The samples were photographed using a Leitz Aristoplan microscope equipped with ep-illuminator and filters for fluorescein isothiocyanate and tetramethyl rhodamine B isothiocyanate fluorescence.

To study the efficiency of a multiple baculovirus infection, immunocytochemical staining of insect cells was used. Sf9 cells were coinfected with two recombinant viruses coding for the α and β subunits of prolyl 4-hydroxylase and immunostained with antibodies to these two subunits (FIG. 3). When the analysis was performed 48 h after infection, 87% of all cells were found to express at least one of the two types of subunit, 90% of cells expressing one type of subunit also expressing the other type.

C. Prolyl 4-Hydroxylase Activity Assay.

The 0.2% Triton X-100 extracts of cell homogenates were analyzed for prolyl 4-hydroxylase activity with an assay based on the hydroxylation-coupled decarboxylation of 2-oxo [1-¹⁴C] glutarate (Kivirikko et al., Methods Enzymol. 82:245-304 (1982)). As reported previously (Veijola et al., J. Biol. Chem. 269:26746-26753 (1994)), a significant level of prolyl 4-hydroxylase activity was found in both Sf9 and High Five cells, the activity in High Five cells being distinctly higher than that in Sf9 cells (Table I). Infection of the cells with a virus coding for the proα1 (III) chains had only minor effects on this activity, whereas the activity in cells infected with the virus coding for the proα1 (III) chain together with viruses coding for the two types of subunit of human prolyl 4-hydroxylase was markedly higher (Table I).

D. Assay For Measuring Collagen.

The amount of the purified type III collagen was determined by using the Sircol collagen assay (Biocolor). Amino acid analysis of the purified type III collagen was performed in an Applied Biosystems 421 Amino Acid Analyzer.

### Example 6

Specifically Engineered Procollagens and Collagens

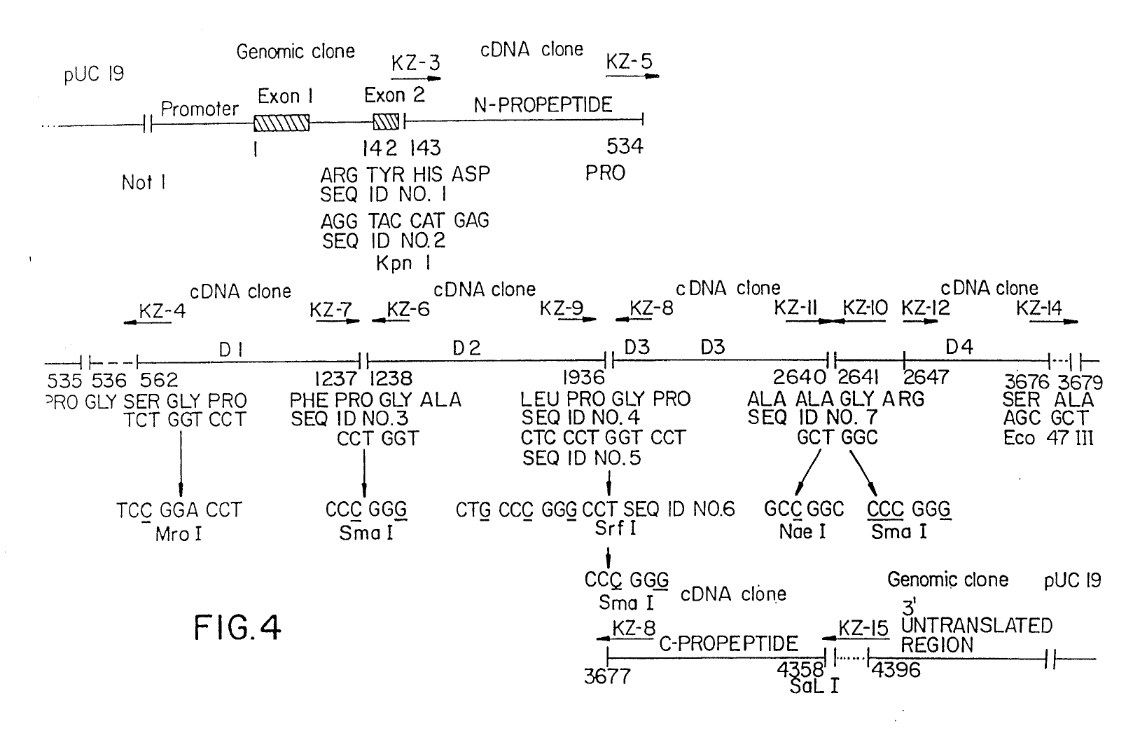

As indicated in FIG. 4, a hybrid gene consisting of some genomic DNA and some cDNA for the proα1(I) chain of human type I procollagen was the starting material. The DNA sequence of the hybrid gene was analyzed and the codons for amino acids that formed the junctions between the repeating D-periods were modified in ways that did not change the amino acids encoded but did create unique sites for cleavage of the hybrid gene by restriction endonucleases.

A. Recombinant procollagen or collagen

The D3-period of proα1(I) is excised using SrfI and NaeI restriction nucleases. The bases coding for the amino acids found in the collagenase recognition site present in the D3 period are modified so that they code for a different amino acid sequence. The cassette is amplified and reinserted in the gene. Expression of the gene in an appropriate host cell will result in type I collagen which cannot be cleaved by collagenase.

B. Procollagen or collagen deletion mutants

A D2 period cassette (of the proα1(I) chain) is excised from the gene described above by digestion with SmaI. The gene is reassembled to provide a gene having a specific 5 in-frame deletion of the codons for the D-2 period.

C. Procollagen or collagen addition mutants

Multiple copies of one or more D-cassettes may be inserted at the engineered sites to provide multiple copies of desired regions of procollagen or collagen.

### Example 7

Expression of Human Prolyl 4-Hydroxylase in a Recombinant DNA System

To obtain expression of the two genes for prolyl 4-hydroxylase in insect cells, the following procedures were carried out. The baculovirus transfer-vector pVlα58 was constructed by digesting a pBluescript (Stratagene) vector containing in the Small site the full-length cDNA for the α subunit of human prolyl 4-hydroxylase, Pα-58 (Helaakoski, et al., Proc. Natl. Acad. Sci. USA 86, 4392-4396 (1989)), with PstI and BamHI, the cleavage sites which closely flank the SmaI site. The resulting Pstl-Pstl and PstI-BamHI fragments containing 61 bp of the 5′ untranslated sequence, the whole coding region, and 551 bp of the 3′ untranslated sequence were cloned to the PstI-BamHI site for the baculovirus transfer vector pVL1392 (Luckow, et al., Virology 170:31-39 (1989)). The baculovirus transfer vector pVLa59 was similarly constructed from pVL1392 and another cDNA clone, Pα-59 (Helaakoski, et al., supra), encoding the α subunit of human prolyl 4-hydroxylase. The cDNA clones Pα-58 and Pα-59 differ by a stretch of 64 bp.

The pVLβ vector was constructed by litigation of an EcoRI-BamHI fragment of a full-length cDNA for the β subunit of human prolyl 4-hydroxylase, S-138 (Pihlajaniemi et al., EMBO J. 6:643-649 (1987)) containing 44 bp of the 5′ untranslated sequence, the whole coding region, and 207 bp of the 3 untranslated sequence to EcoRI/BamHI-digested pVL1392. Recombinant baculovirus transfer vectors were cotransfected into Sf9 cells (Summers et al., Tex. Agric. Exp. St. Bull. 1555:1-56 (1987)) with wild-type Autographa californica nuclear polyhedrosis virus (AcNPV) DNA by calcium phosphate transfection. The resultant viral pool in the supernatant of the transfected cells was collected 4 days later and used for plaque assay. Recombinant occlusion-negative plaques were subjected to three rounds of plaque purification to generate recombinant viruses totally free of contaminating wild-type virus. The screening procedure and isolation of the recombinant viruses essentially followed by the method of Summers and Smith, supra. The resulting recombinant viruses from pVLα58, pVLα59, and pvLβ were designated as the α58 virus, α59 virus and β virus, respectively.

Sf9 cells were cultured in TNM-FH medium (Sigma) supplemented with 10% fetal bovine serum at 27° C. either as monolayers or in suspension in spinner flasks (Techne). To produce recombinant proteins, Sf9 cells seeded at a density of 10⁵ cells per ml were injected at a multiplicity of 5-10 with recombinant viruses when the α58, α59, or β virus was used alone. The α and β viruses were used for infection in ratios of 1:10-10:1 when producing the prolyl 4-hydroxylase tetramer. The cells were harvested 72 hours after infection, homogenized in 0.01 M Tris, pH 7.8/0.1 M NaCl/0.1 M glycine/10 μM dithiothreitol/0.1% Triton X-100, and centrifuged. The resulting supernatants were analyzed by SDS/10% PAGE or nondenaturing 7.5% PAGE and assayed for enzyme activities. The cell pellets were further solubilized in 1% SDS and analyzed by SDS/10% PAGE. The cell medium at 24-96 hours postinfection was also analyzed by SDS/10% PAGE to identify any secretion of the resultant proteins into the medium. The cells in these experiments were grown in TNM-FH medium without serum.

When the time course of protein expression was examined, Sf9 cells infected with recombinant viruses were labeled with [³⁵S]methionine (10 μCi/μl; Amersham; 1 Ci=37CBq) for 2 hours at various time points between 24 and 50 hours after infection and collected for analysis by SDS/10% PAGE. To determine the maximal accumulation of recombinant protein, cells were harvested at various times from 24 to 96 hours after infection and analyzed on by SDS/10% PAGE. Both the 0.1% Triton X-100- and 1% SDS-soluble fractions of the cells were analyzed. Prolyl 4-hydroxylase activity was assayed by a method based on the decarboxylation of 2-oxo[1-¹⁴C]glutarate (Kivirikko et al., Methods in Enzymology 82:245-304 (1982)). The Km values were determined by varying the concentrations of one substrate in the presence of fixed concentration of the second, while the concentrations of the other substrates were held constant (Myllyla et al., Eur. J. Biochem. 80:349-357 (1977)). Protein disulfide-isomerase activity of the β subunit was measured by glutathione: insulin transhydrogenase assay (Carmichael et al., J. Biol. Chem. 252:7163-7167 (1977)). Western blot analysis was performed using a monoclonal antibody, 5B5, to the β subunit of human prolyl 4-hydroxylase (Hoyhtya et al., Eur. J. Biochem. 141:477-482 (1984)). Prolyl 4-hydroxylase was purified by a procedure consisting of poly (L-proline) affinity chromatography, DEAE-cellulose chromatography, and gel filtration (Kivirikko et al., Methods in Enzymology 144:96-114 (1987)).

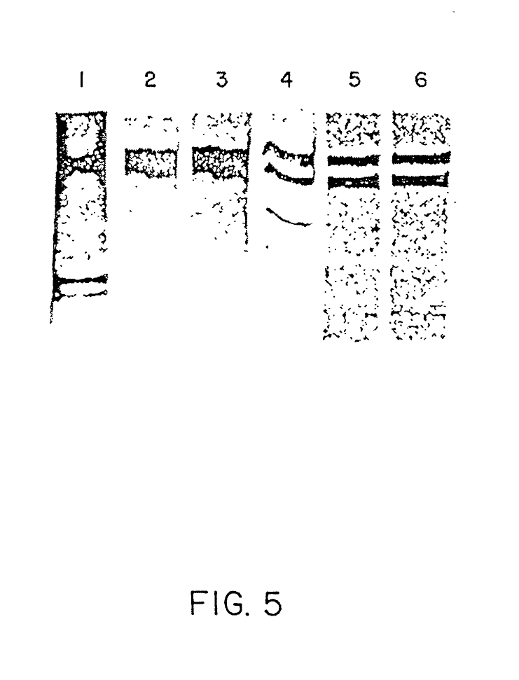

FIG. 5 presents analysis of the prolyl 4-hydroxylase synthesized by the insect cells after purification of the protein by affinity-column chromatography. When examined by polyacrylamide gel electrophoresis in a nondenaturing gel, the recombinant enzyme co-migrated with the tetrameric and active form of the normal enzyme purified from chick embryos. After the purified recombinant enzyme was reduced, the α- and β- subunits were detected. As set forth in FIG. 5, lanes 1-3 are protein separated under non-denaturing conditions and showing tetramers of the two kinds of subunits. Lanes 4-6 are the same samples separated under denaturing conditions so that the two subunits appear as separate bonds.

Table 2 presented data on the enzymic activity of the recombinant enzyme. The Km values were determined by varying the concentration of one substrate in the presence of fixed concentrations of the second while the concentration of the other substrates were held constant.

TABLE 2

Km value, μM

| Substrate | α58₂β₂ | α59₂β₂ | Chick enzyme |
|---|---|---|---|
| Fe⁺² | 4 | 4 | 4 |
| 2-oxoglutarate | 22 | 25 | 22 |
| ascorbate | 330 | 330 | 300 |
| (Pro-Pro- | 18 | 18 | 15-20 |
| Gly)₁₀ | | | |

As indicated, the Michales-Mento (Km) values for the recombinant enzyme were essentially the same as for the authentic normal enzyme from chick embryos.

Since the transfected insect cells synthesize large amounts of active prolyl 4-hydroxylase, they are appropriate cells to transfect with genes of the present invention coding for procollagens and collagens so as to obtain synthesis of large amounts of the procollagens and collagens. Transfection of the cells with genes of the present invention is performed as described in Example 3.

### Example 8

Expression of Recombinant Collagen Genes in Saccharomyces cerevisiae Yeast Expressing Recombinant Genes for Prolyl 4-Hydroxylase

The yeast Saccharomyces cerevisiae can be used with any of a large number of expression vectors. One of the most commonly employed expression vectors is the multi-copy 2μ plasmid that contains sequences for propagation both in yeast and E. coli, a yeast promoter and terminator for efficient transmission of the foreign gene. Typical examples of such vectors based on 2μ plasmids are pWYG4 that has the 2μ ORI-STB elements, the GALI romoter, and the 2μ D gene terminator. In this vector an Ncol cloning site is used insert the gene for either the α or β subunit of prolyl 4-hydroxylase, and provide the ATG start codon for either the α or β subunit. As another example, the expression vector can be pWYG7L that has intact 2μ ORI, STB, REP1 and REP2, the GAL7 promoter, and uses the FLP terminator. In this vector, the gene for either the α or β subunit of prolyl 4-hydroxylase is inserted in the polylinker with its 5′ ends at a BamHI or Ncol site. The vector containing the prolyl 4-hydroxylase gene is transformed into S. cerevisiae either after removal of the cell wall to produce spheroplasts that take up DNA on treatment with calcium and polyethylene glycol or by treatment of intact cells with lithium ions. Alternatively, DNA can be introduced by electroporation. Transformants can be selected by using host yeast cells that are auxotrophic for leucine, tryptophane, uracil or histidine together with selectable marker genes such as LEU2, TRO1, URA3, HIS3 or LEU2-D. Expression of the prolyl 4-hydroxylase genes driven by the galactose promoters can be induced by growing the culture on a non-repressing, non-inducing sugar so that very rapid induction follows addition of galactose; by growing the culture in glucose medium and then removing the glucose by centrifugation and washing the cells before resuspension in galactose medium; and by growing the cells in medium containing both glucose and galactose so that the glucose is preferentially metabolized before galactose-induction can occur. Further manipulations of the transformed cells are performed as described above to incorporate genes for both subunits of prolyl 4-hydroxylase and desired collagen or procollagen genes into the cells to achieve expression of collagen and procollagen that is adequately hydroxylated by prolyl 4-hydroxylase to fold into a stable triple helical conformation and therefore accompanied by the requisite folding associated with normal biological function.

### Example 9

Expression of Recombinant Collagen Genes in Pichia pastoris Yeast Expressing Recombinant Genes for Prolyl 4-Hydroxylase

Expression of the genes for prolyl 4-hydroxylase and procollagens or collagens can also be in non-Saccharomyces yeast such as Pichia pastoris that appear to have special advantages in producing high yields of recombinant protein in scaled-up procedures. Typical expression in the methylotroph P. pastoris is obtained by the promoter from the tightly regulated AOX1 gene that encodes for alcohol oxidase and can be induced to give high levels of recombinant protein driven by the promoter after addition of methanol to the cultures. Since P. Pastoris has no native plasmids, the yeast is employed with expression vectors designed for chromosomal integration and genes such as HIS4 are used for selection. By subsequent manipulations of the same cells, expression of genes for procollagens and collagens described herein is achieved under conditions where the recombinant protein is adequately hydroxylated by prolyl 4-hydroxylase and, therefore, can fold into a stable helix that is required for the normal biological function of the proteins in forming fibrils.

### Example 10

Expression of Recombinant Collagen Genes in Insect Cells Expressing Recombinant Genes for Prolyl 4-Hydroxylase

A. Construction of Recombinant Vectors Containing Collagen Genes.

pVLC1A1: The baculovirus transfer vector was constructed using the eukaryotic expression vector CMV-COL1A1 (Geddis et al., Matrix 13:399-405 (1993)) and the polyhedrin-based baculovirus transfer vector pVL 1392 (Luckow et al., Virology 170:31-39 (1989)). CMV-COL1A1 contains the sequences coding for the full length cDNA sequence of the α1 chain of the human procollagen I (COL1A1). Digestion of CMV-COL1A1 with XbaI generates the full length cDNA for COL1A1 including six bp 5′ untranslated, and 222 bp 3′ untranslated, and this fragment is cloned into the XbaI site of pVL1392 to give the plasmid pVLC1A1.

pVLC1A2: The baculovirus transfer vector was constructed using the vector pVC-HP2010 (Kuivaniem et al., Biochem. J. 252:633-640 (1988)) and the polyhedrin-based baculovirus transfer vector pVL 1392 (Luckow et al., Virology 170:31-39 (1989)). pVC-HP2010 contains the sequences coding for the full length cDNA sequence of the α2 chain of the human procollagen I (COL1A2) in the SphI site of pUC19. PVC-HP2010 is digested with SphI, the GTAC overhang is removed with T4 DNA Polymerase, and the blunt ended fragment is cloned into the EcoRV site of pSP72 (Promega). A BglII site is made six bp upstream of the translation initiation site by PCR to give the plasmid pSP72-ClA2T, and the full length cDNA for COL1A2 is generated by cutting pSP72-C1A2T with BglII-BamHI. The BglII-BamHI fragment from pSP72-C1A2T has the full length COL1A2 sequence plus six bp 5′ untranslated, and 278 bp 3′ untranslated, and this fragment is cloned into the BglII-BamHI sites of pVL1392 to give pVLC1A2.

pVLC3Al: A BglII site was created 16 bp upstream of the translation initiation codon to a full-length cDNA including 92 bp 5′ untranslated region and 715 bp 3′ untranslated region for the proα1 chain of human type III procollagen in the plasmid pBS-SM38 (derived from sequences presented in Ala-Kokko et al. Biochem. J. 260: 509-516 (1989), and GenBank accession number X14420) by PCR to give the plasmid pBS-C3A1. pBS-C3A1 was digested with BglII and XbaI restriction enzymes and the Bglll/Xbal fragment containing the full-length cDNA of proα1 chain of human type III procollagen including 16 bp 5′ untranslated region, and 715 bp 3′ untranslated region, was then ligated to pVL1392 (Luckow et al. Virology 170:31-39 (1989)) to give the plasmid pVLC3A1.

pVLC3A15′UT/C2A1: The baculovirus transfer vector was constructed using the sequences presented in Baldwin et al., Biochem. J. 262:521-528 (1989) resulting in the vector pGEMC2A1 and the polyhedrin-based baculovirus transfer vector pVL 1392 (Luckow et al., Virology 170:31-39 (1989)). pGEMC2A1 contains the sequences coding for exon I from type I collagen, and type II collagen starts from exon 2B. pGEMC2A1 is digested with XbaI-DraI to generate a fragment with the full length cDNA fusion, and six bp 5′ untranslated region and 396 bp 3′ untranslated region, and this fragment is cloned into the XbaI-SmaI sites of pVL1392 to give the plasmid pVLC1A1/C2A1. The 5′ untranslated region was then changed to GATCTGATATT by cloning into the BglII-XbaI sites of the COL II vector.

pVLC3A1NP/C2A1: pGEMC2A1 is digested with XbaI-BamHI and the full length cDNA fusion is cloned into the XbaI-BamHI sites of pBS(SK-) to give the plasmid pBSClAl/C2A1. pBSC1A1/C2A1 is digested with BglII-NarI to generate a full length cDNA without the N-propeptide, the N-propeptide with 16 bp 5′ untranslated from type III collagen was synthesized by PCR and the 35 bp fragment of telopeptide from type II collagen was synthesized by oligonucleotides (chemical synthesis), and these fragments were ligated into pBSC1A1/C2A1 digested with BglII-NarI. This hybrid full length cDNA was excised with BglII-DraI and cloned into the BglII-NotI (the NotI site is blunt ended) sites of pVL1392 to give the plasmid pVLC3A1NP/C2A1.

pVLC4A1: The baculovirus transfer vector was constructed using the vector α1CMVC which was constructed by R. Niecht Köln (based on the sequence published by Brazel et al., Eur. J. Biochem. 168:529-536 (1987), and Soininen et al., FEBS Lett. 225:188-194 (1987)) and the polyhedrin-based baculovirus transfer vector pVL 1392 (Luckow et al., Virology 170:31-39 (1989)). α1CMVC was digested with ClaI to generate a full length cDNA with 18 bp 5′ untranslated and 203 bp 3′ untranslated, and this fragment was blunt ended using Klenow polymerase (Pharmacia Biotech) and a mixture of dNTPS- and cloned into the SmaI site of pVL1392 to give the plasmid pVLC4A1.

pVLE26: The baculovirus transfer vector was constructed using the cDNA E-26 in vector pBluescript (SK-) (Pihlajaniemi et al., J. Biol. Chem. 265:16922-16928 (1990)) and the polyhedrin-based transfer vector pVL.1392 (Luckow et al., Virology 170:31-39 (1989)). The cDNA E-26 encodes the α1 chain of human type XIII collagen and it is ligated into the EcoR1 site of pBS(SK-) (construct termed clone E-26). Clone E-26 is digested with EcoR1 to generate the E-26 cDNA covering type XIII coding sequences. 123 bp 5′ untranslated region and 117 bp 3′ untranslated region are included, and this fragment is cloned into the EcoR1 site of pVLl92 to give the plasmid pVLE26.

pVLhuXIII: The baculovirus transfer vector was constructed using clone E-26 (Pihlajaniemi et al., J. Biol. Chem. 265:,16922-16928 (1990)), genomic human type XIII collagen sequences (Tikka et al., J. Biol. Chem. 266:17713-17719 (1991)) and the polyhedrin-based baculovirus transfer vector pVL1932 (Luckow et al., Virology 170:31-39 (1989) ). A clone called pBShuXIII was constructed and it contains the clone E-26 of the α1 chain of human type XIII collagen with the 5′ end of genomic human type XIII collagen generated by PCR, in the NotI-EcoR1 site of the pBS(SK-) to give the full-length cDNA of type XIII collagen. In pBSHuXIII the 5′ end of the genomic human type XIII collagen is generated by PCR and it covers nucleotides 1-272 from the type XIII collagen gene (Tikka et al., J. Biol. Chem. 266:17713-17719 (1991)) The 5′-PCR-primer included a new NotI restriction site preceding the type XIII sequences, which was used as well as a PstI site between nucleotides 216 and 217 (Tikka et al., J. Biol. Chem. 266:17713-17719 (1991)), when cloning the 5′-PCR-product into the clone E-26 digested with NotI cleaving at the pBluescript (SK-) polylinker site and with PstI digesting between nucleotides 78 and 79 (Pihlajaniemi et al., J. Biol. Chem. 265:16922-16928 (1990)). pBShuXIII is digested with NotI-EcoR1 to generate the full-length cDNA with 10 bp 5′ untranslated region and 117 bp 3′ untranslated region, and this fragment is cloned into the NotI-EcoR1 sites of pVL1392 to give the plasmid pVLhuXIII.

pVLmoXIII: The baculovirus transfer vector was constructed using the vector pBSmoXIII and the polyhedrin-based baculovirus transfer vector pVLl392, which is described in Luckow et al., Virology 170:31-39 (1989). pBSmoXIII consists of a clone encoding the α1 chain of mouse type XIII collagen wherein the 5′ and 3′ ends were generated by PCR using the cDNA sequence for mouse α1 chain of type XIII collagen, and ligated in the EcoR1 site of the pBS(SK-) to give the full-length cDNA of type XIII collagen. Specifically, the following oligonucleotides were used as primers for the PCR reaction:

| | |
|---|---|
| 1. 5′ ATGAATTCAAGTTCTACTCGCGTAGGCGC 3′ | (nt 767-787); |
| 2. 5′ ATGAATTCCCGAAGATGTCTCCAGGATGT 3′ | (nt 796-817); |
| 3. 5′ ATGAATTCAAGGGTCAGTGTGGAGAGT 3′ | (nt 1121-1139); |
| 4. 5′ TTGAATTCGTGTGGGTACTCTCCACACTGACC 3′ | (complementary to nt 1124-1147); |
| 5. ATGAATTCCTGCCTCCTCCGATGGCATT 3′ | (complementary to nt 1614-1636); |
| 6. 5′ ATGAATTCGCCTCCAGGAATGAAGGGAGAAGT 3′ | (complementary to nt 2047-2070); |
| 7. 5′ ATGAATTCGTTCCAGCAGCCTTGGACTGGTAAGC 3′ | (complementary to nt 2661-2686); |
| 8. 5′ ATGAATTCGCCAGTCCCAGGTTAGAGGCA 3′ | (complementary to nt 2693-2713). |

pBSmoXIII covers the sequences from nucleotide 466 to 857 and from nucleotide 2350 to 2926 of the cDNA sequence for mouse α1 chain of type XIII collagen ligated to the BbsI site (in the COL1 domain) and to the StuI site (in the COL3 domain) of the clone. pBSmoXIII is digested with EcoR1 to generate a full-length type XIII collagen variant with seven base pairs 5′ untranslated and 288 base pairs 3′ untranslated, and this fragment was cloned into the EcoR1 site of pVL1392 to give the plasmid pVLmoXIII. Another alternatively spliced full-length cDNA variant for the α1 chain of mouse type XIII collagen was constructed and is termed pVLmoXIII(+E12). This construction is identical to pVLmoXIII, except that it includes also the sequence that encodes exon 12.

pVLC15A1: The baculovirus transfer vector was constructed a PCR fragment covering nucleotides 14 to 1374 (Kivirikko et al., J. Biol. Chem. 269: 4773-4779, (1994)) and containing an EcoRV linker sequence at the 5′ and an EcoRI linker sequence at the 3′ end of the fragment ligated into the EcoRV-EcoRI site of pBluescript (SK-). This construct was digested by Sphl (cleaving in the PCR fragment at sequences corresponding to nucleotide 1355 of sequences presented in Kivirikko et al., J. Biol. Chem. 269:4773-4779 (1994) and EcoRI digesting at the polylinker of the pBluescript. An Sphl-EcoRI fragment of clone SK5-3 covering nucleotides 1355-4330 in Kivirikko et al., J. Biol. Chem. 269:4773-4779 (1994), was ligated to the above Sphl EcoRI digested construct with the PCR fragment resulting in construct pBShuXV. pBShuXV is digested with EcoRV (cleaving at pBluescript polylinker) and Hincll (cleaving at nucleatide 4309 of type XV collagen cDNA sequences) to generate the full length cDNA for COL XV including 76 bp 5′ untranslated region, and 53 bp 3′ untranslated region, and this fragment is cloned in the Smal site of pV11392 (Luckow et al., Virology 170:31-39 (1989)) to give the plasmid pVLCL5A1.

M18K: The baculovirus transfer vector was constructed using the vectors pBsSXT-5B5, pBsMM-21.3 and pBsMM-103 (Rehn et al., J. Biol. Chem. 270:4705-4711 (1995)) which were used to generate pBluescript SV M18kok.11 (pBsM18kok.11), and the polyhedrin-based baculovirus transfer vector pVL 1393 (Invitrogen). pbluescript SK M18kok.11 contains the shortest variant of the α1 chain of mouse type XVIII collagen (1315 amino acid residues). pBsM18kok.11 is digested with EcoRV-NotI to generate the full length cDNA including 22 bp 5′ untranslated region and 180 bp 3′ untranslated region, and this fragment is cloned into the SmaI-NotI sites of PVL1393 to give the plasmid M18K.

M18VA2K: The baculovirus transfer vector was constructed using the vectors pBsM18kok.11 and pBsV2.5, which contains the long NC1, NC1-764 domain (Rehn et al., J. Biol. Chem. 270:4705-4711 (1995)), to generate pBsM18VA2 and the polyhedrin-based baculovirus transfer vector pVL 1393 (Invitrogen). Several steps were performed in order to build the ensuing cDNA construct pBsM18VA2K from the sequence info in the published article. pBsM18VA2K was digested with EcoRV-NotI to generate full length cDNA including 3 bp 5′ untranslated region and 180 bp 3′ untranslated region, and this fragment is cloned into the SmaI-NotI sites of pVL 1393 to give the plasmid M18VA2K.

M18VA2N: The baculovirus transfer was constructed using the vector pBluescript SK COL XVIII, encoding the NC1-301 (Rehn et al., Proc. Nat'l. Acad. Sci 91: 4234-4238 (1994)), and the vector pBs V2.5, encoding the NC1-764 (Rehn et al., J. Biol. Chem. 270:4705-4711 (1995)), and the polyhedrin-based baculovirus transfer vector pVL 1393 (Invitrogen). The plasmid pBsM18VA2N contains the cDNA for the N-terminal noncollagenous domain of the shortest variant of the α1 chain of mouse type XVIII collagen. pBsM18VA2N is mutated by PCR to generate a translation termination codon at nucleotides 1691-1693. pBsM18VA2N is digested with EcoRV/NotI to generate the cDNA of the NC1-764 and 3 bp 5′ untranslated region. This fragment is cloned into the Smal-Notl sites of pVI1393 to give the plasmid M18VA2N.

M18NC1: The baculovirus transfer vector was constructed using the vector pBluescript SK COL XVIII NC1 (Rehn et al., Proc. Natl. Acad. Sci. USA 91:4234-4238 (1994)) and the polyhedrin-based baculovirus transfer vector pVL 1393 (Invitrogen). pBluescript SK COL XVXVIII NC1 contains the cDNA for the N-terminal noncollagenous domain of the shortest variant of the α1 chain of mouse type XVIII collagen (1315 amino acid residues). pBluescript SK COL XVIII NC1 is mutated by PCR to generate a stop codon at the 3′ end of the NC1 domain. pBsM18NC1 is digested with EcoRV-NotI to generate the cDNA of the NC1 domain and 22 bp 5′ untranslated, this fragment is cloned into the SmaI-NotI sites of pVL1393.

M18C: The baculovirus transfer vector was constructed using the vector pbluescript SK MM-103 (Rehn et al., J. Biol. Chem. 269:13929-13935 (1994)) and the polyhedrin-based baculovirus transfer vector pVL 1393 (Invitrogen). pBluescript SK MM-103 contains the cDNA for the C-terminus of the α1 chain of mouse type XVIII collagen in the NotI site of pBluescript SK. pBluescript SK MM-103 digested with EcoRI-NotI which generates a cDNA fragment covering nucleotides 2802-4080 (see, Rehn et al., J. Biol. Chem. 269:13929-13935 (1994)) with a translation initiation codon at nucleotides 3010-3012 corresponding to the C-terminal noncollagenous domain (amino acid residues 997-1315) with 180 bp of the 3′ untranslated region, this fragment is cloned into the EcoRI-NotI sites of the pVL 1393 to give M18C.

B. Construction of Recombinant Vectors Containing Collagen Modifying Enzymes.

pVLβ: The baculovirus transfer vector was constructed using the vector pSB(sr)5138 which contains the full length cDNA for human prolyl 4-hydroxylase β-subunit in the EcoRI site (Pihlajaniemi et al., EMBO, J. 6:643 (1987)) and the polyhedrin-based baculovirus transfer vector pVL 1392. pSB(sr)5138 was digested with EcoRI-BamHI to generate the full length cDNA plus 44 bp 5′ untranslated and 207 bp 3′ untranslated, and this fragment was cloned into the EcoRI-BamHI sites of pVL1392 (Vuori et al., Proc. Natl. Acad. Sci. USA 89:7467-7470 (1992)) to give the plasmid pVLβ

pVLα: The baculovirus transfer vector was constructed using the vector pBS-PA59 which contains the full length cDNA for human prolyl 4-hydroxylase α-subunit in the SmaI site (Helmkoski et al.,, Proc. Nat'l. Acad. Sci. USA 86:4392-4396 (1989)) and the polyhedrin-based baculovirus transfer vector pVL 1392. pBS-PA59 was digested with PstI and BamHI to generate PstI-PstI and PstI-BamHI fragments containing the full length cDNA plus 61 bp 5′ untranslated region, and 551 bp 3′ untranslated region, and these fragments are cloned into the PstI-BamHI sites of pVL1392 (Vuori et al., Proc. Natl. Acad. Sci. USA 89:7467-7470 (1992)) to give the plasmid pVLα.

p2Bacαβ: pBS(SK-)S138 was digested with BamHI to give the full length β-subunit of human prolyl 4-hydroxylase including 44 bp 5′ untranslated region and 207 bp 3′ untranslated region. This fragment was cloned into the BamHI site of p2Bac to give p2Bacβ.

pBS(SK-)PA59 was mutated by PCR to place a NotI site 46 bp upstream of the initiation codon for the α-subunit of prolyl 4-hydroxylase to give the plasmid pBS-PA59/5′UTNotI. pBS-PA59/5′UTNotI is digested with NotI to generate a fragment with the full length α-subunit of prolyl 4-hydroxylase including 46 bp 5′ untranslated region and 3 bp 3′ untranslated region. This fragment is cloned into the NotI site of p2Bacβ to give the plasmid p2Bacαβ.

C. Expression of Recombinant Collagen Genes in Insect Cells with Prolyl-4-Hydroxylase.

Recombinant human collagens I, II, III, IV, XIII, XV, and XVIII have been expressed in insect cells by means of baculovirus expression vectors.

Expression of Collagen Type III. pVLC3A1 is a recombinant expression vector encoding the full proα1 chain of human type III collagen. Similar baculovirus expression vectors pVLα, pVLβ, and p2Bacαβ were created for the expression of human prolyl 4-hydroxylase in insect cells. The constructs were transfected in various combinations into insect cells using a BaculoGold™ transfection kit (Pharmigen).

Insect cells (sf9 or High Five, Invitrogen) were cultured in TNM-FH medium (Sigma) supplemented with 10% fetal bovine serum (BioClear) or in a serum-free HyQ CCM3 medium (HyClone) either as monolayers or in suspension in shaker flasks at 27° C. To produce recombinant proteins, insect cells seeded at a density 5−6×10⁵/ml were infected at a multiplicity of 5-10 with the recombinant virus and at a multiplicity of 1 with the viruses for the α subunit and β subunit of human prolyl 4-hydroxylase (Vuori et al., Proc. Natl. Acad. Sci. USA 89:7467-7470 (1992)). Ascorbate (80 μg/ml) was added daily to the culture medium. The cells were harvested 48-120 h after infection, washed with a solution of 0.15 M NaCl and 0.02 M phosphate, pH 7.4, homogenized in a 0.3 M NaCl, 0.2% Triton X-100 and 0.07 M Tris buffer, pH 7.4, and centrifuged at 10,000×g for 20 min. The remaining cell pellet that was insoluble in the homogenization buffer was further solubilized in 1% SDS and analyzed by SDS-PAGE¹. The cell culture medium was concentrated 10 times in an ultrafiltration cell (Cmicon) with a PM-100 membrane. Aliquots of the supernatants of the cell homogenates and the concentrated cell culture medium were analyzed by denaturing SDS-PAGE, followed by staining with Coomassie Brilliant Blue or Western blotting with an antibody to the N-propeptide of human type III procollagen.

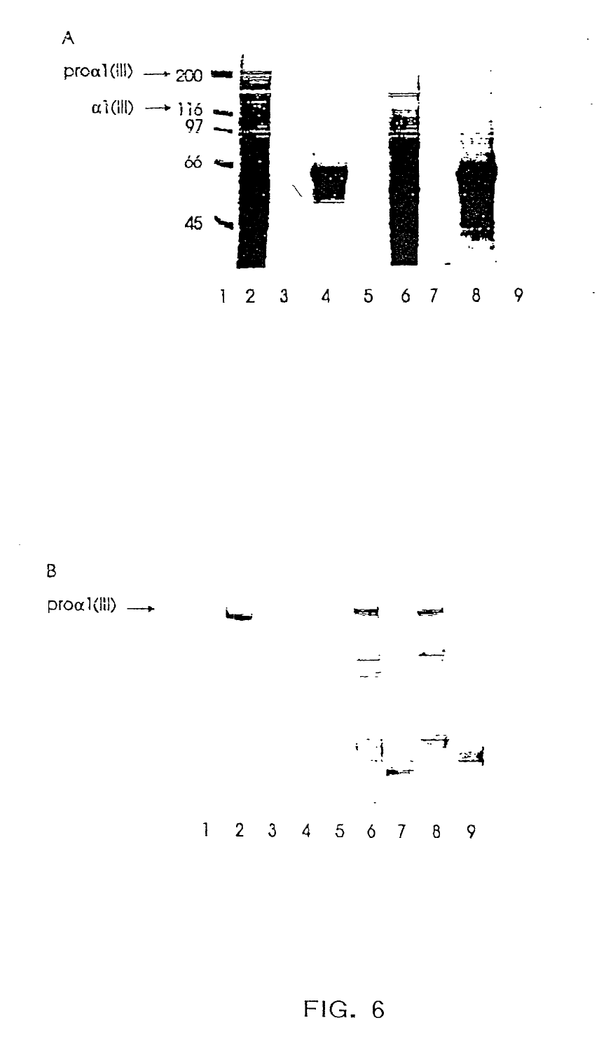

More specifically, Sf9 and High Five cells were infected with a recombinant baculovirus coding for the proα1 (III) chains, harvested 72 h after infection, homogenized in a buffer containing 0.2% Triton X-100 and centrifuged. Aliquots of the Triton X-100 soluble protein fraction and the concentrated cell culture medium were then analyzed either without pepsin treatment of after treatment with pepsin for 1 h at 22° C. The samples were electrophoresed on 8% SDS-PAGE and analyzed by Coomassie staining in A and by Western blotting using an antibody to the N-propeptide of human type III procollagen in B. As set forth in FIG. 6, Lane 1 sets forth molecular weight markers; lanes 2-3, cell extracts; and lanes 4-5, media from Sf9 cell cultures; lanes 6-7, cell extracts; and lanes 8-9, media from High Five cell cultures. Samples in the odd numbered lanes were digested with pepsin. Because the antibody used in the Western blotting reacts only with the N-propeptide of type III procollagen, it does not recognize pepsin digested samples. The arrows indicate the proα1 (III) and α1 (III) chains.

Other aliquots were studied by a radioimmuno assay for the trimeric N-propeptide of human type III procollagen (Farmos Diagnostica) and a colorimetric method for 4-hydroxyproline (Kivirikko et al., Anal. Biochem. 19:249-255 (1967)). Still further aliquots were digested with pepsin for 1 h at 22° C.(Bruckner et al., Anal. Biochem. 110:360-368 (1981)), and the thermal stability of the pepsin-resistant recombinant type III collagen was measured by rapid digestion with a mixture of trypsin and chymotrypsin.

The expression level of proα1 (III) could be seen by Western blotting in samples of the Triton X-100 soluble proteins (FIG. 6B, lanes 2 and 6) and cell culture media (FIG. 6B, lanes 4 and 8) in both Sf9 and High Five cells. After the pepsin digestion the α1 chains of type III collagen were seen in the High Five cells in the Coomassie stained gel (FIG. 6A, lane 7). The pepsin resistant α1(III) chains were not detected in the Western blot (FIG. 6B, lanes 3, 5, 7 and 9) since the antibody used reacts only with the N-propeptides of the proα1 (III) chains, which were apparently digested by pepsin.

Sf9 and High Five cells were infected with the virus coding for the proα1 (III) chains either with or without viruses coding for the two types of subunit of prolyl 4-hydroxylase (Table III). The expression level of total type III procollagen was measured with a radioimmuno assay for the trimeric N-propeptide, and the amount of 4-hydroxyproline formed in the cells was determined by a colorimeric assay. Both values were used to calculate the amount of type III collagen produced by assuming that all the proα1 (III) chains formed triple-helical molecules and that all the hydroxylatable proline residues in the proα1 (III) chains had been converted to 4-hydroxyproline. Based on the known structure of type III procollagen and the amount of 4-hydroxyproline in type III collagen, the amount of type III collagen in the samples was calculated by multiplying the N-propeptide values obtain by 7 and the 4-hydroxyproline values by 8. All measurements were made 72 h after the infection.

A considerable variation was found in the values obtained in different experiments as shown in Table II. Notwithstanding this variation, Table II provides: First, the amount of 4-hydroxyproline formed was in all experiments distinctly higher in cells infected with the prolyl 4-hydroxylase-coding viruses than in their absence. Second, the expression level obtained in High Five cells was consistently higher than that obtained in Sf9 cells. Third, in cells coinfected with the prolyl 4-hydroxylase-coding viruses the level of type III collagen produced was always higher when calculated from the 4-hydroxyproline values than from the radioimmuno assay values, suggesting either that some of the N-propeptides of type III procollagen were degraded or that some of the fully 4-hydroxylated proα1 (III) chains remained nontriple-helical. The highest type III collagen expression values were in the High Five cells that also expressed prolyl 4-hydroxylase, the amount of cellular type III collagen in these cells being about 41-81 μg/5×10⁶ cells (Table III). The amount of type III collagen secreted into the culture medium, when measured with the radioimmuno assay, was about 25-50% of total in Sf9 cells and about 10-30% of total in High Five cells.

Experiments were also performed in which High Five cells were grown in suspension in shaker flasks. A similar effect of prolyl 4-hydroxylase-coding viruses was seen in these experiments as above. The highest expression levels found in such experiments have ranged up to about 40 mg of type III collagen produced per liter of culture in 72 h, about 80-90% of the collagen produced being found in the cell pellet, and 10-20% in the medium.

TABLE III
Prolyl 4-hydroxylase activity of Triton X-100 extracts
from insect cells expressing proα1 chains of human type
III procollagen with or without the α and β subunits of
prolyl 4-hydroxylase.

| | Prolyl 4-hydroxylase |
|---|---|
| Cells and recombinant | activity |
| polypeptides expressed | dpm/10 μl |
| High Five cells | |
| None | 480 |
| Proα1 (III) chains | 500 |
| Proα1 (III) chains and α | 4810 |
| and β subunits | |
| Sf9 cells | |
| None | 150 |
| Proα1 (III) chains | 60 |
| Proα1 (III) chains and α | 3360 |
| and β subunits | |

The cells expressed either no recombinant polypeptide or only the proα1 (III) chains or the latter plus the α and β subunits of prolyl 4-hydroxylase. The analysis was performed 72 h after the infection.
The values are given as dpm/10 μl of the Triton extract, mean of duplicate values obtained in three experiments for High Five cells, and mean of duplicate values in one experiment for Sf9 cells.

Expression of Collagen Types I and II. Baculovirus expression vectors pVLC1A1 and pVLC1A2 were created for the expression of the proα1 chain and the proα2 chain of human collagen I, and pVLC3A15′UT/C2A1 was created for the expression of the proα1 chain of human collagen II.

Unless otherwise specified, insect cells were cultured, and recombinant collagen produced following the procedures supra.

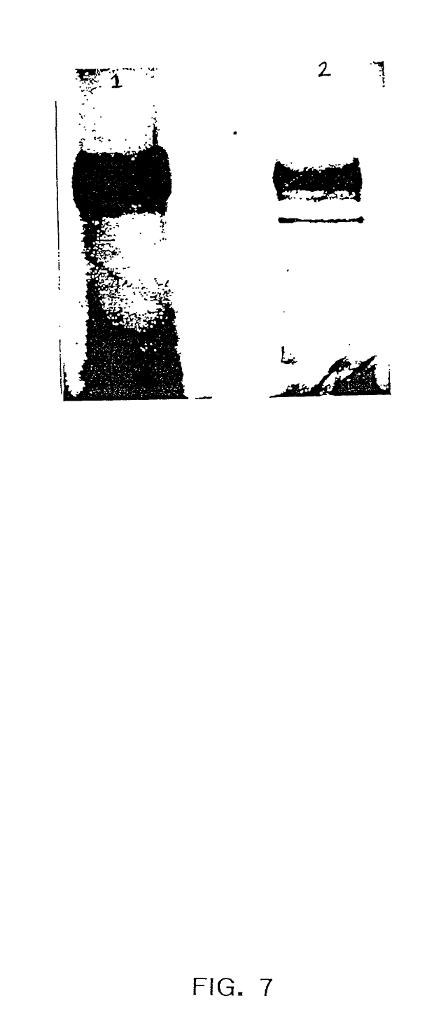

The expression level of proα1 (I), and proα1 (I) and proα2 (I) in the presence of prolyl 4-hydroxylase, and following pepsin digestion of the supernatants from cell homogenates could be seen in silver-stained 5% SDS-PAGE. See FIG. 7, lanes (DIA 1). The silver-stained SDS PAGE revealed the formation of triple-helical procollagen I in these cells. Homotrimeric collagen can be separated from heterotrimeric collagen I on a metal chelate affinity column through the use of a histidine-tag to the C-terminal domain of the proα2 chain.

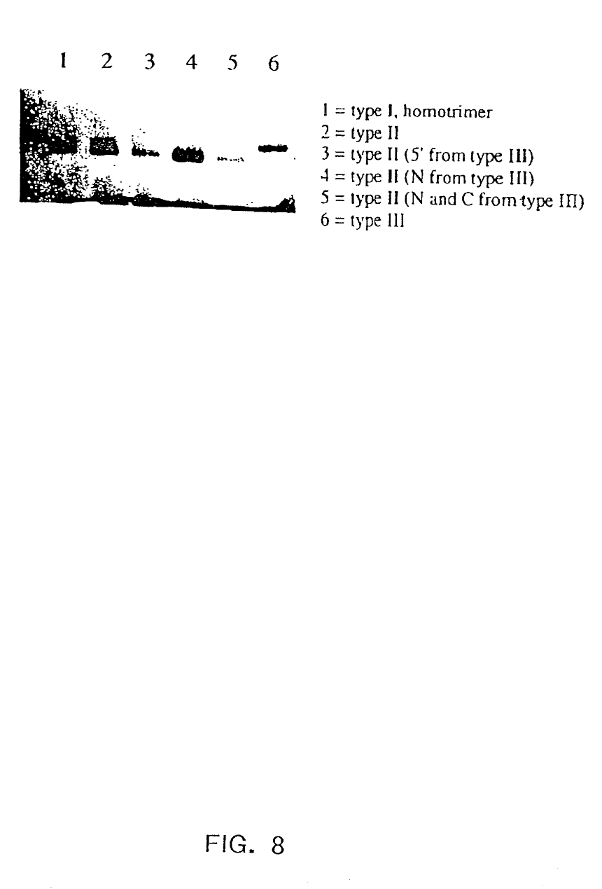

The expression level of proα1 (II) in the presence of prolyl 4-hydroxylase could be seen in coomassie stained 5% SDS PAGE. See FIG. 8 (wherein lane 1 depicts the expression of a homotrimer of type I collagen; lane 2 is a standard sample of type II procollagen; lane 6 is a standard sample of type III procollagen; and lanes 3-5 compare three different constructs of human type II procollagen containing varying amounts of human procollagen type III. Lane 3 is type II procollagen with the C-terminal end of type III procollagen; lane 4 is type II procollagen with the N-terminal non-collagenous region from type III procollagen; and lane 5 is type II procollagen with the N- and C-terminal regions of type III procollagen).

Several baculovirus vectors for the expression of human type II collagen were constructed. In one of these vectors, the 5′ untranslated region of human type II collagen was replaced with human type III collagen 5′ untranslated region. In another vector, the entire human type II collagen gene was expressed. In another insect expression vector, the N-propeptide of type II collagen was replaced with an N-propeptide of type III collagen. All three of those vectors were found to express human type II collagen in varying levels. Expression was detected by Coomassie Blue stain SDS-PAGE and by Western blot analysis.

Expression of Collagen Types IV, XIII, and XVIII. pVLC4A1 is a recombinant baculovirus expression vector encoding the proα1 chain of human collagen IV. pVLhuXIII is a recombinant baculovirus vector encoding the proα1 chain of human collagen XIII. pVLC15A1 is a recombinant expression vector encoding the proα1 chain of human collagen XV. M18K and M18VA2K are recombinant expression vectors encoding two variants of the proα1 chain of human collagen type XVIII.

Unless otherwise specified, insect cells were cultured and recombinant collagen produced following the procedures supra. pVLC4A1, pVLhuXIII, pVLC15A1, M18K, and M18VA2K have been transformed into insect cells, and the recombinant collagens have been successfully expressed.

D. Purification And Analysis Of Recombinant Collagen.

Purification of Recombinant Type III Collagen. The properties of the purified human type III collagen produced in insect cells were found to be very similar to those of the type III collagen extracted from carious tissues (Kielty et al., Connective Tissue and Its Heritable Disorders: Molecular, Genetic and Medical Aspects pp. 103-147 (1993); Kivirikko, Ann. Med. 25:113-125 (1993); van der Rest et al., Adv. Mol. Cell. Biol. 6:1-67 (1993); Brewton et al., Extracellular Matrix Assembly and Structure pp. 129-170 (1994); Pihlajaniemi et al., Prog. Nucleic Acid Res. Mol. Biol. 50:225-262 (1995); Prockop et al., Annu. Rev. Biochem. 64:403-434 (1995)). In particular, the content of 4-hydroxyproline and the Tₘ of the triple helices, when determined by CD spectra, were found to be virtually identical to those of the authentic type III collagen. The content of hydroxylysine in the recombinant collagen was found to be about one-half of that of type III collagen extracted from various tissues, indicating that insect cells must have a considerable level of lysyl hydroxylase activity.

Insect cells expressing the recombinant type III procollagen were washed with a solution of 0.15 M NaCl and 0.02 M phosphate, pH 7.4, homogenized in a cold 0.2 M. NaCl, 0.1% Triton X-100 and 0.05 M Tris buffer, pH 7.4 (20×10⁶ cells/ml), incubated on ice for 30 min, and centrifuged at 16,000×g for 30 min. Unless otherwise mentioned, all the following steps were performed at 4° C. The supernatant was chromatographed on a DEAE cellulose column (DE-52, Whatman) equilibrated and eluted with a 0.2 M NaCl and 0.05 M Tris buffer, pH 7.4, the void volume being collected. The pH of the sample was lowered to 2.0-2.5, and the sample was digested with a final concentration of 150 μg/ml of pepsin for 1 h at 22° C. Pepsin was irreversibly inactivated by neutralization of the sample followed by an overnight incubation on ice. The recombinant type III collagen was precipitated by adding solid NaCl to a final concentration of 2 M and centrifugation at 16,000×g for 1 h. The pellet was dissolved in a 0.5 M NaCl, 0.5 M urea, and 0.05 M Tris buffer, pH 7.4, for 1 day, and the sample was digested with pepsin as above for a second time. The sample was then chromatographed on a Sephacryl HR-500 gel filtration column (Pharmacia), eluted with a solution of 0.2 M NaCl and 0.05 M Tris, pH 7.4, dialyzed against 0.1 M acetic acid and lyophilized.

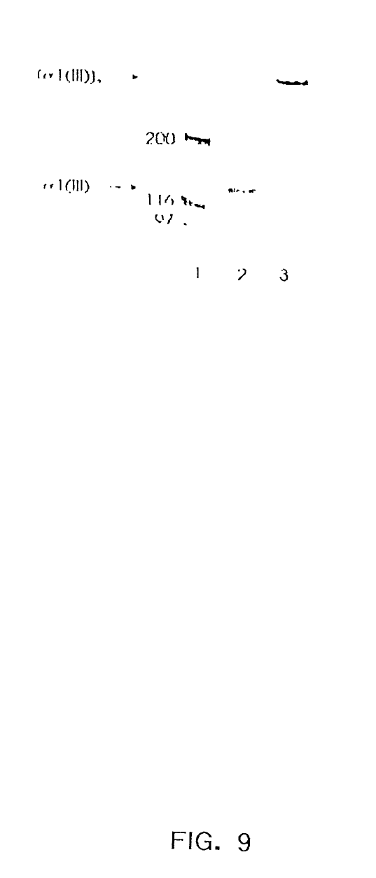

Type III procollagen was expressed in High Five cells cultured either as monolayers or in suspension in shaker flasks. The cells were harvested 72 h after infection, homogenized in a buffer containing 0.1% Triton X-100 and centrifuged, and the supernatant of the cell homogenate was passed through a DEAE cellulose column to remove nucleic acids. The flow through fractions containing the type III procollagen were pooled and digested with pepsin. This converted the type III procollagen to type III collagen and digested most of the noncollagenous proteins. The type III collagen was then concentrated by salt precipitation, solubilized and treated with pepsin as above. The type III collagen was finally separated from pepsin and other remaining contaminants by gel filtration on a Sephacryl S 500-HR column. The fractions containing the type III collagen were pooled, dialyzed and lyophilized. The purified type III collagen was analyzed by 5% SDS-PAGE under reducing (FIG. 9, lane 2) and nonreducing (FIG. 9, lane 3) conditions. No contaminants were seen in the Coomassie stained gel and the type III collagen α1 chains were disulfide-bonded. Amino acid and CD spectrum analysis were performed on the purified type III collagen. The amino acid composition of the recombinant type III obtained corresponded well with the amino acid composition reported for human type III collagen. The only exception was the amount of hydroxylysine, which was 3 residues/1000 amino acids in the recombinant type III collagen instead of 5/1000 amino acids in the authentic human type III collagen. The melting temperature of the recombinant type III collagen determined by CD spectrum analysis was 40° C.

The High Five cells gave consistently higher production rates than Sf9 cells, the highest production rates seen in High Five cells cultured in monolayers ranging up to about 80 μg of cellular recombinant human type III collagen/5×10⁶ cells, which corresponds to about 120 Mg of type III procollagen. When the High Five cells were cultured--in suspension in shaker flasks, the highest amount of cellular type III collagen produced ranged up to about 40 mg/l, corresponding to about 60 mg/l of type III procollagen.

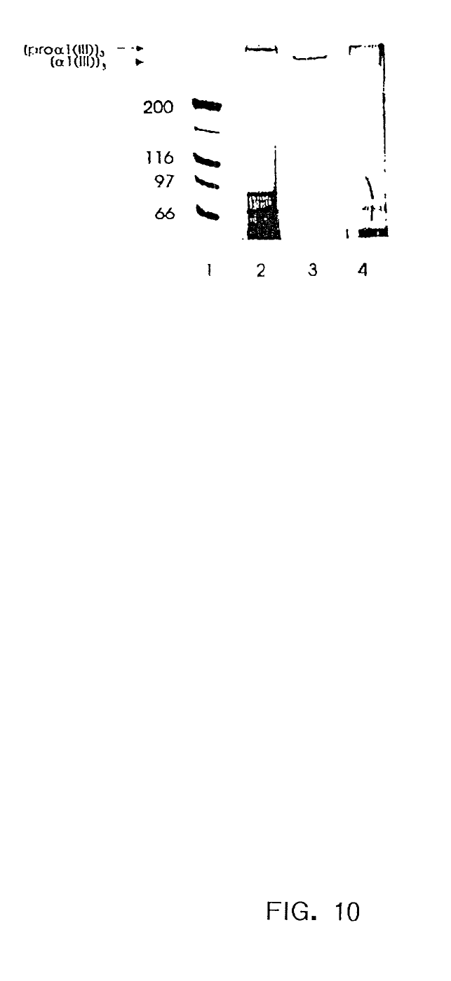

Conformational Integrity of the Recombinant Type III Collagen. Association of the proα1 (III) chains into trimers was studied by using SDS-PAGE analysis under nonreducing conditions. High Five cells were coinfected with viruses coding for the proα1 (III) chains and the α and β subunits of human prolyl 4-hydroxylase. The cells were harvested 72 h after infection, homogenized in a buffer containing 0.2% Triton X-100, centrifuged, and the remaining cell pellets were further solubilized in 1% SDS. Aliquots of the Triton soluble proteins were treated with pepsin for 1 h at 22° C. Essentially all the proα1 (III) chains synthesized were found as disulf ide-bonded trimers based on the disappearance of a protein band of a high molecular weight (FIG. 10, lane 2). After pepsin digestion the band corresponding to the recombinant type III procollagen was converted to a band corresponding to type III collagen, and the protein remained in the form of the trimer, thus indicating the existence of disulf ide bonds between the α1 (III) chains (FIG. 10, lane 3). Virtually all the type III procollagen expressed was soluble in the Triton X-100-containing homogenization buffer, as no band corresponding to type III procollagen was seen in the Triton X-100-insoluble, SDS-soluble fraction (FIG. 10, lane 4).

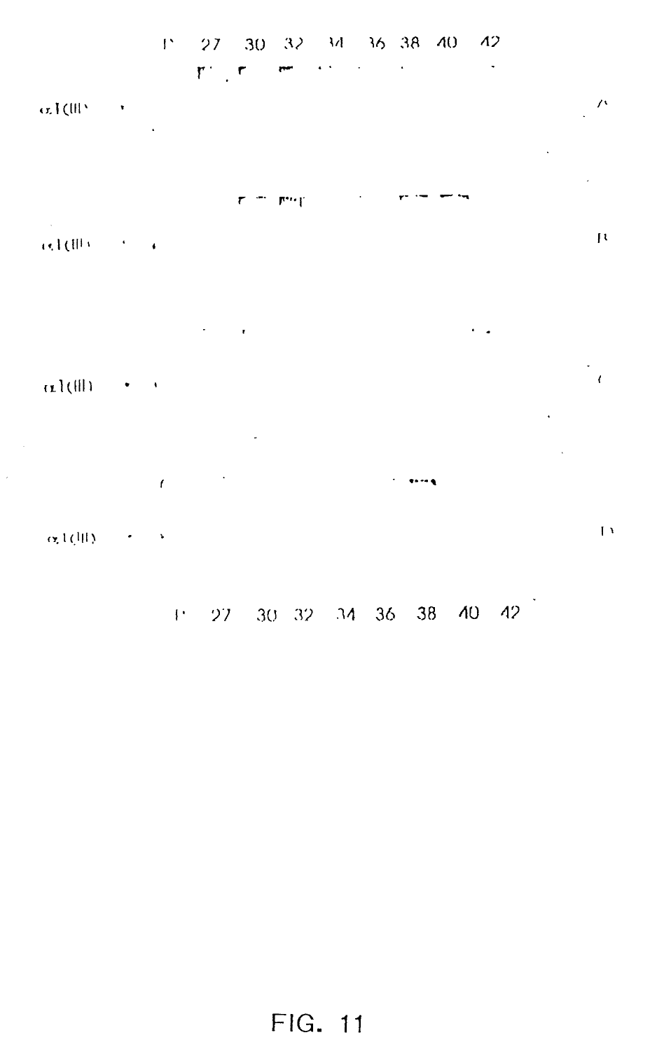

The thermal stability of the type III collagen expressed under different cell culture conditions was studied by using digestion with a mixture of trypsin and chymotrypsin after heating to various temperatures (Bruckner, et al., Anal. Biochem. 110:360-368 (1981)). High Five cells were infected with viruses coding for the proα1 (III) chains and the α and β subunits of human prolyl 4-hydroxylase. The cells were harvested 72 h after infection, homogenized in a buffer containing 0.2% Triton X-100 and centrifuged. In these experiments, ascorbate was either added daily to the cell culture medium as usual or omitted during the infection. The Triton X-100 soluble proteins were first digested with pepsin for 1 h at 22° C. to convert type III procollagen to type III collagen (Pihlajaniemi et al., EMBO J. 6:643-649 (1987)), and the trypsin/chymotrypsin digestion was then performed for aliquots of the pepsin-treated samples. The samples were then electrophoresed on 8% SDS-PAGE and analyzed by Coomassie staining. FIG. 11 provides the results of this thermal stability for a variety of collagen products. As set forth in panel A, the cells were infected only with the virus coding for the proα1 (III) chains, and ascorbate was omitted from the culture medium; panel B, the cells were infected only with the virus coding for the proα1 (III) chains, and ascorbate was present in the culture medium as usually; panel C, the cells were coinfected with viruses coding for the proα1 (III) chains, and the α and β subunits of prolyl 4-hydroxylase, but ascorbate was omitted from the culture medium; and panel D, the cells were infected with the three viruses, and ascorbate was present in the culture medium. Lane P shows a sample digested with pepsin without subsequent trypsin/chymotrypsin digestion, lanes 27-42 show samples treated with the trypsin/chymotrypsin mixture at the temperatures indicated. The arrows show the position of the αl (III) chains. As evidenced by these results, when the proα1 (III) chains were expressed without the presence of prolyl 4-hydroxylase and ascorbate, the Tₘ of type III collagen was found to be at about 32-34° C. (FIG. 11A). The presence of either ascorbate of prolyl 4-hydroxylase without the other had virtually no increasing effect on the thermal stability (FIGS. 11B and 11C). In contrast, when the proα1 (III) chains were produced in the presence of both prolyl 4-hydroxylase and ascorbate, the Tₘ of type III collagen was increased considerably, being at about 38-40° C. (FIG. 11D).

Purification and analysis of Collagen Types I and II. Collagens types I and II were purified as described supra. The recombinant type II human collagen expressed from the recombinant insect cells was found to exhibit resistance to trypsin and chymotrypsin digestion. These protease digestion experiments indicated that triple helical type II human collagen was formed in the recombinant insect cells.

The thermal stability of the recombinant type II human collagen expressed from the recombinant insect cells was measured and compared with native type I human collagen. These results indicated that the recombinant type II collagen had a triple helical structure. The Tₘ of the recombinant type II collagen was up to about 40° C.

### Example 11

Expression of Recombinant Collagen Genes in Yeast Cells Expressing Recombinant Genes for Prolyl 4-Hydroxylase

A. Construction of Recombinant Vectors Containing Collagen Genes.

pPIC9ColIII. This plasmid contains the human Col III gene joined to the α-mating factor secretion signal (α-MFSS) (and containing a deletion of the native human secretion signal).

The 3′ end of the COL III gene was synthesized by PCR from the 4195 bp downstream (EcoRI site) of the translation initiation codon to the stop codon (4401 bp). NotI and XbaI sites were created in the 3′ end of the PCR fragment. The fragment was digested with EcoRI and XbaI and cloned into the EcoRI and XbaI sites of pBluescript-SM38 (pBS-SM38 is derived from sequences presented in Ala-Kokko et al. Biochem. J. 260: 509-516 (1989)), and GenBank accession number X14420) to give the plasmid pBluescript-SM38/B.

The 5′ end of the Col III gene was synthesized from 73 bp downstream of the translation initiation codon to 176 bp (BamHI site) by PCR (for sequences, see Ala-Kokko et al., Biochem., J. 260:509-516 (1989)), and ClaI and NotI sites were created in the 5′ end of the PCR fragment. pBluescript-SM38/B was digested with ClaI and BamHI, and the two fragments from this digest and the 5′ PCR fragment were ligated with T4 ligase to give the plasmid pBluescript-SM38/11.

pBluescript-SM38/11 was digested by NotI and the NotI-NotI collagen fragment (73-4401 bp) was cloned in frame with the α-factor signal sequence in the yeast expression vector pPIC9 (Invitrogen) to give the plasmid pPIC9COLIII.

pHII-D2/colIII. The 3′ end of the COL III gene was synthesized by PCR from the 4195 bp downstream (EcoRI site) of the translation initiation codon to the stop codon (4401 bp) by PCR using pBluescript-SM38. An XbaI site was created in the 3′ end of the PCR fragment. pBluescript-C3A1 was digested with EcoRI and XbaI and the large fragment isolated, and the 3′ PCR fragment is digested with EcoRI and XbaI. These two fragments are ligated with T4 ligase to give pBluescript-C3A1/10. A BglII site was created 16 bp upstream of the translation initiation codon in pBluescript-C3A1/10 and the BglII-XbaI fragment from pBluescript-C3A1/10, contianing collagen sequences from (nucleotides −16 to 4401) is ligated into the EcoRI site of pHIL-D2 (Invitrogen) to give plasmid PHII-D2/colIII.

pAO815β. pYM25 was digested with HpaI and the fragment containing the ARG4 gene of Saccharomyces cerevisiae was isolated and cloned into the EcoRV sites of pAO815 (Invitrogen) replacing the HIS5 gene with ARG4, to give the plasmid pARG815.

A cDNA of the β subunit of human prolyl 4-hydroxylase (Vuori et al., Proc. Nat'l. Acad. Sci. USA 89:7467-7470 (1992)) was synthesized by PCR from the translation initiation codon to the stop codon by PCR, and EcoRI sites were created in the 5′ and 3′ ends of the PCR fragment. The C-terminal endoplasmic reticulum retention peptide -KDEL- was modified to the Yeast ER retention signal -HDEL- by PCR. This PCR fragment was digested with EcoRI and cloned into pBluescript SK, to give pbluescript SKβ/20. pBluescript SKβ/20 was digested with EcoRI and this fragment was cloned into the EcoRI site of pAO815 (Invitrogen), to give the plasmid pAO815β which has a single expression cassette for the β-subunit of prolyl 4-hydroxylase.

pARG815α. The 5′ end of the α-subunit of prolyl 4-hydroxylase was synthesized by PCR from the translation initiation codon to the 689 bp downstream (HindIII site), and HindIII and SmaI sites were created in the 5′ end of the fragment. pA-59 (Vuori et al., Proc. Nat'l. Acad. Sci. USA 89:7467-7470 (1992)) was digested with HindIII and the large fragment was isolated and ligated with the 5′ PCR fragment to give pA-59/15.

The 3′ end of the α-subunit was synthesized by PCR from 1373 bp (PstI site) downstream of the translation initiation codon to the translation stop codon, and SmaI and BamHI sites were created in the 3′ end of the fragment. pA-59/15 was digested with PstI and BamHI, and the large fragment was isolated, and ligated with the 3′ PCR fragment to give pA-59/3. pA-59/3 was digested with SmaI and the SmaI-SmaI α-subunit fragment was cloned into the EcoRI site of pARG815, to give pARG815α.

pARG815αβ. pAO815β was digested with BglII and BamHI to excise the expression cassette, and the expression cassette is cloned into the BamHI site of pARG815α to give the vector pARG815αβ.

pAO815αββ—is similar to pAO815αβ, but contains two cassettes of the β subunit of the human prolyl 4-hydroxylase gene. pAO815β was digested with BglII and BamHI to excise the expression cassette, and the expression cassette is cloned into the BamHI site of pARG815αβ to give the vector pARG815αββ.

The β-subunit without its signal sequence was synthesized by PcR from 52 bp downstream of the translation initiation codon to the translation stop codon. EcoRI restriction sites were created in 5′ and 3′ ends. This PCR fragment was cloned into the EcoRI site of pSP72 (Promega).

The Pichia pastoris host strain used for the expression was obtained from Dr. james Cregg. The strain has two auxotrophic mutations his4 and arg4.

B. Expression of Recombinant Collagen Genes in Yeast Cells with Prolyl-4-Hydroxylase.

Pichia pastoris host strain GS115 was stably transformed with combinations of the plasmid described supra and related plasmids to produce the following recombinant strains.

P. pastoris Col IIIαβ—carries the human Col III gene with α-MFSS and both subunits of the human Prolyl 4-hydroxylase.

P pastoris nCol III—is similar to P. pastoris nCol III αβ, but uses the native Col III signal sequence.

P. pastoris αβ—carries both subunits of human prolyl 4-hydroxylase.

P. pastoris αββ contains human prolyl 4-hydroxylase, wherein the α:β gene ration is 1:2.

P. pastoris α contains the human prolyl 4-hydroxylase α gene.

P. pastoris β contains the human prolyl 4-hydroxylase β gene.

The P. pastoris strains described in paragraph 5 were grown in rotary shakers to an OD₆₀₀ of 5.0. Samples were taken and run on PAGE gels. Western blots were performed and analyzed with antibodies against procol III N-terminal peptide, the α-subunit of human prolyl 4-hydroxylase and the β-subunit of human prolyl 4-hydroxylase.

The Western blots described in paragraph 6 demonstrated that both human collagen III and human prolyl 4-hydroxylase were produced in P. pastoris.

Pepsin digestion experiments were performed to test for triple helical structure in the human collagen produced in P. pastoris. Whereas most proteins are degraded by the proteolytic enzyme pepsin, the triple helical region of collagen is pepsin resistant. The collagen from cell lysates of P. pastoris Col IIIαβ were digested with pepsin, and the digestion products were separated by SDS-PAGE. The results of these experiments indicated that triple helical human collagen III was produced in the recombinant P. pastoris cells.

Experiments were performed to measure human prolyl 4-hydroxylase activity in the P. pastoris strains described above. P. pastoris has no intrinsic prolyl 4-hydroxylase activity. The assay were performed with ¹⁴C labelled proline, essentially as described by Kivirikko in Methods in Enzymology, Volume 82, pgs. 245-304, Academic Press, San Diego, Calif. Prolyl 4-hydroxylase activity was found in the recombinant cells.

### Example 12

### Expression of Recombinant Collagen Genes in Mammalian Cells Expressing Recombinant Genes for Prolyl 4-Hydroxylase

A. Construction of a Recombinant Semliki Forest Virus Vectors Containing Collagen Genes.

pSFVmoXIII: The Semliki Forest expression vector was constructed using the vector pBSmoXIII generated based on clones and sequences as described for pVLmoXIII above (Rehn et al., submitted; Peltonen et al., submitted) and the eukaryotic expression vector pSFV-1 (Liljeström et al., Bio/tecnology 9:1356-1361 (1991)). pBSmoXIII is digested with EcoRI to generate the full-length type XIII collagen variant with seven bp 5′ untranlsated region and 288 bp 3′ untranslated region, and this fragment is made blunt ended with Klenow, and cloned into the SmaI site of pSFV-1 to give the plasmid pSFVmoXIII. pSFVmoXIII plasmid was used to produce RNA by in vitro transcription using MEGAscript in vitro transcription kit by Ambion. Baby hamster kidney (BNK) cells transfected with the RNA as described in Lilegestrom et al., Current Protocols in Molecular Biology 2:16-20 (1991). Synthesis of full-length chains for mouse type XIII collagen were observed in the BHK cells by Western blotting of SDS-polyacrylamide gel-fractionated cell extracts.

Efficient expression of other collagen genes in cells of higher eukaryotes will be based on the above-described Semliki Forest virus vector. Semliki Forest virus is preferred as the virus because it has a broad host range such that infection of the above mentioned mammalian cell lines will also be possible. More specifically, it is expected that the use of the Semliki Forest virus can be used in a wide range of hosts, as the system is not based on chromosomal integration, and therefore it will be a quick way of obtaining modifications of the recombinant collagens in studies aiming at identifying structure-function relationships and testing the effects of various hybrid molecules. In addition, it is expected that use of the Semliki Forest virus will yield very high recombinant expression levels, over 10 μg/1×10⁶ cells.

HeLa cells and the vaccinia virus-based expression system can also be used to express collagens in mammalian cells, and will preferably be used to expresst type IV collagens as homo- and hetero-trimer isoforms of the six type IV collagen chains.

All patents, patents applications, and publications cited are incorporated herein by reference.

The foregoing written specification is considered to be sufficient to enable one skilled in the art to practice the invention. Indeed, various modifications of the above-described makes for carrying out the invention which are obvious to those skilled in the field of immunology, biochemistry, or related fields are intended to be within the scope of the following claims.

1. A method for producing a collagen polypeptide, wherein said collagen is selected from the group comprising collagen types IV, V, VI, VII, VIII, IX, X, XI, XII, XIII, XIV, XV, XVI, XVII, XVIII, and XIX, comprising:
a. culturing a host cell, wherein said host cell has been infected, transfected or transformed with (i) a first expression vector comprising a polynucleotide molecule having a nucleic acid sequence which encodes a collagen subunit; and (ii) a second expression vector comprising a polynucleotide molecule having a nucleic acid sequence which encodes at least one collagen post-translational enzyme or subunit thereof; and
b. purifying said collagen polypeptide.

2. The method of claim 1 wherein the host cell is selected from the group consisting of a yeast cell, a plant cell, an insect cell and a mammalian cell.

3. The method of claim 1 wherein the host cell is further infected, transfected or transformed with a third expression vector comprising a polynucleotide molecule having a nucleic acid sequence which encodes a second collagen subunit.

4. The method of claim 3 wherein the host cell is further infected, transfected or transformed with a fourth expression vector comprising a polynucleotide molecule having a nucleic acid sequence which encodes a third collagen subunit.

5. The method of claim 1 wherein said collagen post-translational enzyme is selected from the group consisting of prolyl-4-hydroxylase, lysyl oxidase, lysyl hydroxylase, C-proteinase, and N-proteinase.

6. The method of claim 1 wherein the collagen post-translational enzyme subunit is selected from the group consisting of an alpha subunit of prolyl-4-hydroxylase and a beta subunit of prolyl-4-hydroxylase.

7. A method for producing a procollagen polypeptide, wherein said procollagen is selected from the group comprising collagen types IV, V, VI, VII, VIII, IX, X, XI, XII, XIII, XIV, XV, XVI, XVII, XVIII, and XIX, comprising:
a. culturing a host cell, wherein said host cell has been infected, transfected or transformed with: (i) a first expression vector comprising a polynucleotide molecule having a nucleic acid sequence which encodes a collagen subunit; and (ii) a second expression vector comprising a polynucleotide molecule having a nucleic acid sequence which encodes at least one collagen post-translational enzyme or subunit thereof; and
b. purifying said procollagen polypeptide.

8. The method of claim 7 wherein the host cell is selected from the group consisting of a yeast cell, a plant cell, an insect cell and a mammalian cell.

9. The method of claim 7 wherein the host cell is further infected, transfected or transformed with a third expression vector comprising a polynucleotide molecule having a nucleic acid sequence which encodes a second collagen subunit.

10. The method of claim 9 wherein the host cell is further infected, transfected or transformed with a fourth expression vector comprising a polynucleotide molecule having a nucleic acid sequence which encodes a third collagen subunit.

11. The method of claim 7 wherein said collagen post-translational enzyme is selected from the group consisting of prolyl-4-hydroxylase, lysyl oxidase and lysyl hydroxylase.

12. The method of claim 7 wherein the collagen post-translational enzyme subunit is selected from the group consisting of an alpha subunit of prolyl-4-hydroxylase and a beta subunit of prolyl-4-hydroxylase.

13. A collagen polypeptide, wherein said collagen is selected from the group comprising collagen types IV, V, VI, VII, VIII, IX, X, XI, XII, XIII, XIV, XV, XVI, XVII, XVIII, and XIX, manufactured according to a method comprising:
a. culturing a host cell, wherein said host cell has been infected, transfected or transformed with: (i) a first expression vector comprising a polynucleotide molecule having a nucleic acid sequence which encodes a collagen subunit; and (ii) a second expression vector comprising a polynucleotide molecule having a nucleic acid sequence which encodes at least one collagen post-translational enzyme or subunit thereof; and
b. purifying said collagen polypeptide.

14. The collagen polypeptide of claim 13 wherein the host cell is selected from the group consisting of a yeast cell, a plant cell, an insect cell and a mammalian cell.

15. The collagen polypeptide of claim 13 wherein the host cell is further infected, transfected or transformed with a third expression vector comprising a polynucleotide molecule having a nucleic acid sequence which encodes a second collagen subunit.

16. The collagen polypeptide of claim 15 wherein the host cell is further infected, transfected or transformed with a fourth expression vector comprising a polynucleotide molecule having a nucleic acid sequence which encodes a third collagen subunit.

17. The collagen polypeptide of claim 13 wherein said collagen post-translational enzyme is selected from the group consisting of prolyl-4-hydroxylase, lysyl oxidase, lysyl hydroxylase, C-proteinase, and N-proteinase.

18. The collagen polypeptide of claim 13 wherein the collagen post-translational enzyme subunit is selected from the group consisting of an alpha subunit of prolyl-4-hydroxylase and a beta subunit of prolyl-4-hydroxylase.

19. The collagen polypeptide of claim 13 wherein said polypeptide is not glycosolated.

20. The collagen polypeptide of claim 13 wherein said polypeptide is partially deglycosolated.

21. A host cell which has been infected, transfected or transformed with: (i) a first expression vector comprising a polynucleotide molecule having a nucleic acid sequence which encodes a collagen subunit; and (ii) a second expression vector comprising a polynucleotide molecule having a nucleic acid sequence which encodes at least one collagen post-translational enzyme or subunit thereof.

22. The host cell of claim 21 wherein said host cell is further infected, transfected or transformed with a third expression vector comprising a second collagen subunit.

23. The host cell of claim 22 wherein said host cell is further infected, transfected or transformed with a fourth expression vector comprising a third collagen subunit.
